(19) United States
(12) Patent Application Publication
(10) Pub. No.: US 2022/0023302 A1
(43) Pub. Date: Jan. 27, 2022

(54) PDE9 INHIBITORS FOR TREATING SICKLE CELL DISEASE

(71) Applicant: Imara Inc., Boston, MA (US)

(72) Inventors: Niels SVENSTRUP, Boston, MA (US); David TISI, Boston, MA (US); Jeffrey WORTHINGTON, Boston, MA (US); Vanik PETROSSIAN, Boston, MA (US)

(21) Appl. No.: 17/493,677

(22) Filed: Oct. 4, 2021

Related U.S. Application Data
(63) Continuation of application No. PCT/US2020/026696, filed on Apr. 3, 2020.
(60) Provisional application No. 62/829,784, filed on Apr. 5, 2019.

(51) Int. Cl. A61K 31/519; A61K 47/26; A61K 47/12; A61K 31/655; A61P 7/06

(52) U.S. Cl. CPC A61K 31/519; A61K 47/26; A61P 7/06; A61K 31/655; A61K 47/12

(57) ABSTRACT

The present disclosure relates to PDE9 inhibitors, pharmaceutical compositions comprising the PDE9 inhibitors, and methods of using the PDE9 pharmaceutical compositions for the treatment of sickle cell disease (SCD).

### CROSS-REFERENCE TO RELATED APPLICATIONS

This application is a continuation of International Application No. PCT/US2020/026696 filed Apr. 3, 2020, which claims the benefit of U.S. Provisional Application No. 62/829,784, filed Apr. 5, 2019, which is incorporated herein by reference in its entirety.

### FIELD OF THE DISCLOSURE

The present disclosure relates to methods of making and using pharmaceutical compositions comprising cyclic guanylate monophosphate (cGMP)-specific phosphodiesterase type 9 inhibitors (hereinafter referred to as PDE9 inhibitors).

### BACKGROUND

Sickle Cell Disease (SCD, also called sickle cell anemia (SCA)) is a genetic disorder leading to vaso-occlusive processes responsible for much of the mortality in SCD patients. SCD disease results from a point mutation in the hemoglobin (HBB) gene producing abnormal sickle hemoglobin (HbS or HbSS), which polymerizes and creates rigid and sticky sickled red blood cells. Sickled red blood cells result in chronic inflammation, elevated cell adhesion, oxidative stress, and endothelial dysfunction culminating in vaso-occlusive processes.

There is to date no cure for SCD. Treatment options include blood transfusion and treatment with the anti-cancer agent hydroxyurea. Blood transfusions correct anemia by increasing the number of normal, non-sickled red blood cells in circulation. Regular transfusion therapy can help prevent recurring strokes in children at high risk. Hydroxyurea (HU) has been approved for the treatment of SCD and shown to reduce the frequency of painful crisis and hospitalization. Unfortunately, HU is often poorly tolerated and its widespread use is limited by concerns about its potential impact on fertility and reproduction; challenges achieving and maintaining an efficacious dose due to its hematologic toxicities; and requirements for monthly monitoring (Heeney et al., Pediatr Clin North Am., 2008, 55(2):483). In fact, it is estimated that only 1 out of 4 adult patients, and possibly even fewer, are treated with this drug (Stettler et al., JAMA, 2015, 313:1671). In addition, many patients are dosed with sub-efficacious doses of HU due to these challenges. Thus, novel, safe, and effective treatments that can be safely employed globally to prevent the morbid complications of SCD in patients of all ages are urgently needed.

There remains a need for treating SCD.

### SUMMARY OF THE DISCLOSURE

The present disclosure provides methods of making and using Compound 1 and/or pharmaceutical compositions comprising Compound 1 or a pharmaceutically acceptable salt, solvate, or hydrate thereof, to treat sickle cell disease.

In one aspect described herein, an oral pharmaceutical composition comprises: about 10 mg/mL of 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or hydrate thereof, and an excipient base comprising about 2.0 mg/mL of potassium sorbate, about 5.0 mg/mL sucralose, and/or about 5.0 mg/mL citric acid, the pharmaceutical composition is in the form of an oral liquid solution suitable for administration to a patient. In some embodiments, the pharmaceutical composition further comprises a flavor. In some embodiments, the flavor is a grape flavor. In some embodiments, the flavor is a raspberry flavor. In some embodiments, the composition further comprises about 3.0 mg/mL of a raspberry flavor.

In another aspect described herein, is a pharmaceutical composition comprising: 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or hydrate thereof; and an excipient base, wherein the composition is in the form of an oral liquid solution. In some embodiments, the pharmaceutical composition comprising from about 5 mg/mL to about 15 mg/mL of Compound 1, or a pharmaceutically acceptable salt, solvate, or hydrate thereof. In some embodiments, the excipient base comprises from about 1.0 mg/mL to about 3.0 mg/mL of potassium sorbate. In some embodiments, the excipient base comprises from about 1.0 mg/mL to about 20.0 mg/mL of sucralose. In some embodiments, the excipient base comprises from about 1.0 mg/mL to about 10.0 mg/mL of citric acid. In some embodiments, the pharmaceutical composition further comprises a flavor. In some embodiments, the flavor is a cherry flavor, a raspberry flavor, a grape flavor, a strawberry flavor, or a tutti-fruity flavor. In some embodiments, the flavor is a grape favor. In some embodiments, the flavor is a raspberry flavor. In some embodiments, the pharmaceutical composition further comprises from about 1.0 mg/mL to about 5.0 mg/mL of a flavor. In some embodiments, the pharmaceutical composition further comprises about 3.0 mg/mL of a raspberry flavor.

In some embodiments, the pharmaceutical composition has a pH from about 3.0 to about 6.0, or from about 5.5 to about 6.5. In some embodiments, the pharmaceutical composition has a pH of about 5.5, about 5.6, about 5.7, about 5.8, about 5.9, about 6.0, about 6.1, about 6.2, about 6.3, about 6.4, or about 6.5. In some embodiments, the pharmaceutical composition has a pH above 5.5.

In another aspect described herein, a method for treating sickle cell disease in a subject in need, comprises administering any of the pharmaceutical compositions above. In some embodiments, the pharmaceutical composition is taken with food. In some embodiments, the pharmaceutical composition is administered once per day, twice per day, or three times per day. In some embodiments, the pharmaceutical composition is administered once per day. In some embodiments, the pharmaceutical composition is administered for at least 4 weeks, 12 weeks, 16 weeks, or 24 weeks. In some embodiments, the method further comprises administering hydroxyurea (HU). In some embodiments, the method comprises administering to the subject about 0.3 mg/kg to about 6.0 mg/kg or from about 0.3 mg/kg to about 1.0 mg/kg of subjects body mass per day or per dose of Compound 1, or a pharmaceutically acceptable salt, solvate, or hydrate thereof. In some embodiments, the patient in need thereof is a pediatric patient.

In another aspect described herein, a method for treating sickle-β⁰ thalassemia in a subject in need, comprises administering any of the pharmaceutical compositions above. In some embodiments, the pharmaceutical composition is taken with food. In some embodiments, the pharmaceutical composition is administered once per day, twice per day, or three times per day. In some embodiments, the pharmaceutical composition is administered once per day. In some embodiments, the pharmaceutical composition is administered for at least 4 weeks, 12 weeks, 16 weeks, or 24 weeks. In some embodiments, the method further comprises administering hydroxyurea (HU). In some embodiments, the method comprises administering to the subject about 0.3 mg/kg to about 6.0 mg/kg or from about 0.3 mg/kg to about 1.0 mg/kg of subjects body mass per day or per dose of Compound 1, or a pharmaceutically acceptable salt, solvate, or hydrate thereof. In some embodiments, the patient in need thereof is a pediatric patient.

### BRIEF DESCRIPTION OF THE DRAWINGS

FIG. 1 shows Compound 1 reduces myeloid and neutrophil inflammatory markers in the lungs of Townes mice.

FIG. 2 shows Compound 1 reduces adhesion of SCD patient neutrophils to endothelial cell lined microfluidic chamber in vitro.

FIG. 3 shows Compound 1 reduces expression of CD11a, CD11b and CD18 integrins on SCD patient neutrophils.

FIG. 4 shows the outcome of studies in the Townes SCD Model comparing Compound 1 (30 mg/kg).

FIG. 5 shows the outcome of studies in the Townes SCD Model comparing Compound 1 (30 mg/kg).

FIG. 6 illustrates a clinical study design for Compound 1.

FIG. 7 depicts, without limitation, a representative sampling of screenshots for use in a mobile device running software designed to track human impact of a pharmaceutical.

FIG. 8 shows the flavor profile of Compound 1 in the original and revised excipient base system with the addition of raspberry flavor.

### DETAILED DESCRIPTION

Phosphodiesterases (PDEs) are a family of enzymes degrading cyclic nucleotides and thereby regulating the cellular levels of second messengers throughout the entire body. PDEs represent attractive drug targets, as proven by a number of compounds that have been introduced to clinical testing and the market, respectively. PDEs are encoded by 21 genes that are functionally separated into 11 families differing with respect to kinetic properties, substrate selectivity, expression, localization pattern, activation, regulation factors and inhibitor sensitivity. The function of PDEs is the degradation of the cyclic nucleotide monophosphates cyclic Adenosine Monophosphate (cAMP) and/or Guanosine Monophosphate (cGMP), which are important intracellular mediators involved in numerous vital processes including the control of neurotransmission and smooth muscle contraction and relaxation.

PDE9 is cGMP specific (Km cAMP is >1000× for cGMP) and is hypothesized to be a key player in regulating cGMP levels as it has the lowest Km among the PDEs for this nucleotide. PDE9 is expressed throughout the brain at low levels with the potential for regulating basal cGMP.

In the periphery, PDE9 expression is highest in prostate, intestine, kidney and haematopoietic cells, enabling therapeutic potential in various non-CNS indications.

In the present disclosure, pharmaceutical compositions comprising PDE9 inhibitors are designed for treatment for Sickle Cell Disease (SCD).

### Compounds of the Disclosure

In the context of the present disclosure, a compound is considered to be a PDE9 inhibitor if the amount required to reach the 50% inhibition level PDE9 is 10 micromolar or less, preferably less than 9 micromolar, such as 8 micromolar or less, such as 7 micromolar or less, such as 6 micromolar or less, such as 5 micromolar or less, such as 4 micromolar or less, such as 3 micromolar or less, more preferably 2 micromolar or less, such as 1 micromolar or less, in particular 500 nM or less. In preferred embodiments the required amount of PDE9 inhibitor required to reach the IC₅₀ level of PDE9 is 400 nM or less, such as 300 nM or less, 200 nM or less, 100 nM or less, or even 80 nM or less, such as 50 nM or less, for example 25 nM or less.

Throughout this application the notations IC₅₀ and IC₅₀ are used interchangeably.

In some embodiments, the PDE9 inhibitor of the present disclosure has low or no blood brain barrier penetration. For example, the ratio of the concentration of a PDE9 inhibitor of the present disclosure in the brain to the concentration of it in the plasma (brain/plasma ratio) may be less than about 0.50, about 0.40, about 0.30, about 0.20, about 0.10, about 0.05, about 0.04, about 0.03, about 0.02, or about 0.01. In some embodiments, the brain/plasma ration is measured 30 min or 120 min after administration of the PDE9 inhibitor.

In some embodiments, the PDE9 inhibitor may be any imidazo pyrazinone PDE9 inhibitor disclosed in WO 2013/053690 and/or any imidazo triazinone PDE9 inhibitor disclosed in WO 2013/110768, the contents of each of which are incorporated herein by reference in their entirety.

In some embodiments, the PDE9 inhibitor is Compound 1 or a pharmaceutically acceptable salt, cocrystal, solvate, hydrate, or polymorph thereof. A racemate form of Compound 1 and an anhydrous form of Compound 1 have been described in WO 2013/053690 and WO 2017/005786. In some embodiments, the PDE9 inhibitor is 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or hydrate thereof. In some Compound 1 has the following structure:

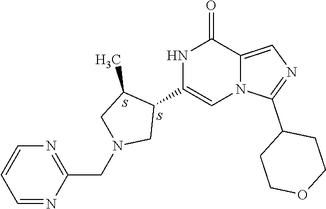

6-[(3 S,4S)-4-methyl-1-(pyrimidin-2-ylmethyl)pyrrolidin-3-yl]-3-tetrahydropyran-4-yl-7H-imidazo[1,5-a]pyrazin-8-one; Formula C₂₁H₂₆N₆O₂; calculated molecular weight about 394 g/mol. In some embodiments, Compound 1 is enantiopure or substantially enantiopure.

### Pharmaceutical Compositions

The present disclosure further provides a pharmaceutical composition comprising a therapeutically effective amount of any of the PDE9 inhibitors and a pharmaceutically acceptable carrier or diluent. In some embodiments, the present disclosure provides a pharmaceutical composition comprising a therapeutically effective amount of Compound 1, or a pharmaceutically acceptable salt or solvate thereof, and a pharmaceutically acceptable carrier or diluent or excipient.

### Pharmaceutically Acceptable Salts

The present disclosure also comprises salts of the PDE9 inhibitors, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in Berge, S. M. et al., J. Pharm. Sci. 1977, 66, 2, the contents of which are hereby incorporated by reference.

Furthermore, the compounds of this disclosure may exist in unsolvated as well as in solvated forms with pharmaceutically acceptable solvents such as water, ethanol, and the like. In some embodiments, the compounds may exist as a hydrate. In general, the solvated forms are considered equivalent to the unsolvated forms for the purposes of this disclosure.

In some embodiments, the pharmaceutical composition comprises Compound 1 as the solvated, unsolvated, or crystalline form. In some embodiments, Compound 1 is present as the unsolvated form. In some embodiments, Compound 1 is present as the present as the crystalline form. In some embodiments, Compound 1 is present as a monohydrate crystalline form. In some embodiments, Compound 1 is presented in the solvated form. In some embodiments, the solvated form is a hydrate form.

### Formulations

The compounds of the disclosure may be administered alone or in combination with pharmaceutically acceptable carriers, diluents or excipients, in either single or multiple doses. The pharmaceutical compositions according to the disclosure may be formulated with pharmaceutically acceptable carriers or diluents as well as any other known adjuvants and excipients in accordance with conventional techniques such as those disclosed in Remington: The Science and Practice of Pharmacy, 22nd Edition, Gennaro, Ed., Mack Publishing Co., Easton, Pa., 2013.

The pharmaceutical compositions may be specifically formulated for administration by any suitable route, such as oral, rectal, nasal, pulmonary, topical (including buccal and sublingual), transdermal, intracisternal, intraperitoneal, vaginal, and parenteral (including subcutaneous, intramuscular, intrathecal, intravenous, and intradermal) routes. It will be appreciated that the route will depend on the general health and age of the subject to be treated, the nature of the condition to be treated, and the active ingredient.

The pharmaceutical compositions of the present invention can be formulated to be compatible with the intended method or route of administration; exemplary routes of administration are set forth herein.

In some embodiments, the pharmaceutical composition is formulated for oral administration to a subject. In some embodiments, the pharmaceutical composition is formulated as a tablet or pill. In some embodiments, the pharmaceutical composition is formulated as a solid tablet suitable for oral administration to a subject. In some embodiments, the pharmaceutical composition is formulated as an oral liquid, solution or suspension suitable for oral administration to a subject.

Pharmaceutical compositions for oral administration include solid dosage forms such as capsules, tablets, dragees, pills, lozenges, powders, and granules. Liquid dosage forms for oral administration include solutions, emulsions, suspensions, syrups, and elixirs, either manufactured as such, or as a solid form for reconstitution prior to use.

In some embodiments, the pharmaceutical composition disclosed herein is in a form for oral dosing. In some embodiments, the pharmaceutical composition in formulated as an aqueous solution or suspension for oral administration.

The present disclosure also provides a process for making a pharmaceutical composition comprising admixing a therapeutically effective amount of a compound of the present disclosure and at least one pharmaceutically acceptable carrier or diluent.

The compounds of this disclosure are generally utilized as the free substance or as a pharmaceutically acceptable salt thereof. Such salts are prepared in a conventional manner by treating a solution or suspension of a compound of the present disclosure with a pharmaceutically acceptable acid. Representative examples of suitable organic and inorganic acids are described above.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of the present disclosure and a pharmaceutically acceptable carrier are then readily administered in a variety of dosage forms suitable for the disclosed routes of administration. The formulations may conveniently be presented in unit dosage form by methods known in the art of pharmacy.

Pharmaceutical compositions of the present disclosure suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a liquid carrier is used, the preparation may be in the form of a syrup, emulsion, soft gelatine capsule or sterile injectable liquid such as an aqueous or non-aqueous liquid suspension or solution.

The pharmaceutical compositions of the disclosure may be prepared by conventional methods in the art. For example, tablets may be prepared by mixing the active ingredient with ordinary adjuvants and/or diluents and subsequently compressing the mixture in a conventional tabletting machine prepare tablets. Examples of adjuvants or diluents comprise: corn starch, potato starch, talcum, magnesium stearate, gelatin, lactose, gums, and the like. Any other adjuvants or additives usually used for such purposes such as colorings, flavorings, preservatives etc. may be used provided that they are compatible with the active ingredients.

The pharmaceutical compositions typically comprise a therapeutically effective amount of Compound 1, or a pharmaceutically acceptable salt, solvate, or hydrate thereof, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

The pharmaceutical compositions of the present invention may also be in the form of an aqueous suspension. Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanthin and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

The pharmaceutical composition comprises PDE9 inhibitor Compound 1. The pharmaceutical composition comprises at least 10%, 20%, 30%, 40%, 50%, 60%, 70%, 80%, or 90% by weight of PDE9 inhibitors of the present disclosure. The pharmaceutical composition comprises at least about 1% to about 90% by weight of PDE9 inhibitors of the present disclosure. The pharmaceutical compositions comprises at least about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. The pharmaceutical compositions comprise at least about 1%, about 10%, about 20%, about 30%, about 40%, about 50%, about 60%, about 70%, about 80%, or about 90%. The pharmaceutical composition comprises at least about 1%, about 10%, about 20%, about 30%, about 40%, about 50%, about 60%, about 70%, or about 80%. The pharmaceutical composition comprises at least at most about 10%, about 20%, about 30%, about 40%, about 50%, about 60%, about 70%, about 80%, or about 90% by weight of PDE9 inhibitors of the present disclosure. The pharmaceutical composition comprises at least about 90% to about 99.9% by weight of PDE9 inhibitors of the present disclosure. The pharmaceutical composition comprises at least about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to about 99.9%, about 91% to about 92%, about 91% to about 93%, about 91% to about 94%, about 91% to about 95%, about 91% to about 96%, about 91% to about 97%, about 91% to about 98%, about 91% to about 99%, about 91% to about 99.9%, about 92% to about 93%, about 92% to about 94%, about 92% to about 95%, about 92% to about 96%, about 92% to about 97%, about 92% to about 98%, about 92% to about 99%, about 92% to about 99.9%, about 93% to about 94%, about 93% to about 95%, about 93% to about 96%, about 93% to about 97%, about 93% to about 98%, about 93% to about 99%, about 93% to about 99.9%, about 94% to about 95%, about 94% to about 96%, about 94% to about 97%, about 94% to about 98%, about 94% to about 99%, about 94% to about 99.9%, about 95% to about 96%, about 95% to about 97%, about 95% to about 98%, about 95% to about 99%, about 95% to about 99.9%, about 96% to about 97%, about 96% to about 98%, about 96% to about 99%, about 96% to about 99.9%, about 97% to about 98%, about 97% to about 99%, about 97% to about 99.9%, about 98% to about 99%, about 98% to about 99.9%, or about 99% to about 99.9%. The pharmaceutical composition comprises at least about 90%, about 91%, about 92%, about 93%, about 94%, about 95%, about 96%, about 97%, about 98%, about 99%, or about 99.9%. The pharmaceutical composition comprises at least about 90%, about 91%, about 92%, about 93%, about 94%, about 95%, about 96%, about 97%, about 98%, or about 99%. The pharmaceutical composition comprises at least at most about 91%, about 92%, about 93%, about 94%, about 95%, about 96%, about 97%, about 98%, about 99%, or about 99.9% by weight of PDE9 inhibitors of the present disclosure. The pharmaceutical composition comprises at least 90%, 91%, 92%, 93%, 94%, 95%, 96%, 97%, 98%, or 99% by weight of PDE9 inhibitors of the present disclosure.

The pharmaceutical composition comprises from about 1 mg/mL to about 50 mg/mL of Compound 1, or a pharmaceutically acceptable salt, solvate, or hydrate thereof. In some embodiments, the pharmaceutical composition comprises from about 1 mg/mL to about 30 mg/mL of Compound 1, or a pharmaceutically acceptable salt, solvate, or hydrate thereof. In some embodiments, the pharmaceutical composition comprises from about 5 mg/mL to about 15 mg/mL of Compound 1, of a pharmaceutically acceptable salt, solvate, or hydrate thereof. In some embodiments, the pharmaceutical composition comprises about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, or about 15 mg/mL of Compound 1, or a pharmaceutically acceptable salt, solvate, or hydrate thereof. In some embodiments, the pharmaceutical composition comprises about 8.0 mg/mL of Compound 1, or a pharmaceutically acceptable salt, solvate, or hydrate thereof. In some embodiments, the pharmaceutical composition comprises about 9.0 mg/mL of Compound 1, or a pharmaceutically acceptable salt, solvate, or hydrate thereof. In some embodiments, the pharmaceutical composition comprises about 10.0 mg/mL of Compound 1, or a pharmaceutically acceptable salt, solvate, or hydrate thereof or a pharmaceutically acceptable salt, solvate, or hydrate thereof or a pharmaceutically acceptable salt, solvate, or hydrate thereof. In some embodiments, the pharmaceutical composition comprises about 11.0 mg/mL of Compound 1, or a pharmaceutically acceptable salt, solvate, or hydrate thereof. In some embodiments, the pharmaceutical composition comprises about 12.0 mg/mL of Compound 1, or a pharmaceutically acceptable salt, solvate, or hydrate thereof.

In some embodiments of the pharmaceutical compositions disclosed herein, Compound 1, or pharmaceutically acceptable salt, solvate, or hydrate thereof, is substantially pure. In some embodiments of the pharmaceutical compositions disclosed herein, Compound 1, or a pharmaceutically acceptable salt, solvate, or hydrate thereof, is substantially free of impurities. In some embodiments of the pharmaceutical compositions disclosed herein, substantially free of impurities is defined as less than about 10.0%, about 5%, about 3.0%, about 1.0%, about 0.5%, about 0.1%, or about 0.05% content of impurities. In some embodiments of the pharmaceutical compositions disclosed herein, substantially free of impurities is defined as less than about 1.0% content of impurities. In some embodiments of the pharmaceutical compositions disclosed herein, substantially free of impurities is defined as less than about 0.5% content of impurities. In some embodiments of the pharmaceutical compositions disclosed herein, substantially free of impurities is defined as less than about 0.1% content of impurities.

In some embodiments of the pharmaceutical compositions disclosed herein, Compound 1, or a pharmaceutically acceptable salt, solvate, or hydrate thereof, is at least about 90%, about 95%, about 98%, or about 99% pure.

In some embodiments of the pharmaceutical compositions disclosed herein, Compound 1, or a pharmaceutically acceptable salt, solvate, or hydrate thereof, is at least about 99.1%, about 99.2%, about 99.3%, about 99.4%, about 99.5%, about 99.6%, about 99.7%, about 99.8%, about 99.9%, or about 100% pure.

In some embodiments, Compound 1 or a pharmaceutically acceptable salt, solvate, or hydrate thereof, is formulated as a pharmaceutical composition for oral administration. For example, it may be in a solid tablet form. The composition for oral administration comprises at least a filler and/or a processing aid. The processing aid may be a glidant or a lubricant. The composition for oral administration may also comprise a coating.

In some embodiments, the pharmaceutical composition comprising Compound 1 or a pharmaceutically acceptable salt, solvate, or hydrate thereof, is stored at controlled room temperature (20-25° C.).

In some embodiments, Compound 1 or a pharmaceutically acceptable salt, solvate, or hydrate thereof, is formulated as a liquid pharmaceutical composition for oral administration. For example, it may be in an oral aqueous or liquid solution or suspension.

In another aspect described herein, is a pharmaceutical composition comprising: 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or hydrate thereof; and an excipient base, wherein the composition is in the form of an oral liquid solution. In some embodiments, the pharmaceutical composition comprises an excipient base. The excipient base may include solubilizers (such as water or propylene glycol), preservatives (including antimicrobial and antioxidant agents), sweeteners, and/or pH modifiers.

Preservative encompass a wide range of antimicrobials and antioxidants. Common preservatives include but are not limited to sorbic acid, sodium sorbate, benzoic acid, sodium benzoate, parabens (such as methyl paraben), lactic acid, propionic acid, isothiazolinones, potassium sorbate, and the like.

In some embodiments, the excipient base comprises a preservative. In some embodiments, the excipient base comprises methyl paraben, sodium benzoate, and/or potassium sorbate.

In some embodiments, the excipient base comprises sodium benzoate and/or methyl paraben. In some embodiments, the excipient base comprises from about 1.0 mg/mL to about 5.0 mg/mL of sodium benzoate and/or methyl paraben. In some embodiments, the excipient base comprises about 2.0 mg/mL of sodium benzoate. In some embodiments, the excipient base comprises about 2.0 mg/mL of methyl paraben. In some embodiments, the excipient base does not comprise sodium benzoate. In some embodiments, the excipient base does not comprise methyl paraben.

In some embodiments, the excipient base comprises potassium sorbate. In some embodiments, the excipient base comprises from about 0.1 mg/mL to about 10.0 mg/mL, or any amount therein of potassium sorbate. In some embodiments, the excipient base comprises from about 1.0 mg/mL to about 5.0 mg/mL of potassium sorbate. In some embodiments, the excipient base comprises from about 1.0 mg/mL to about 3.0 mg/mL of potassium sorbate. In some embodiments, the excipient base comprises about 1.0 mg/mL, 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, or about 3.0 mg/mL of potassium sorbate. In some embodiments, the excipient base comprises about 2.0 mg/mL of potassium sorbate.

In some embodiments, the excipient base comprises from about 0.01% to about 0.5% w/v of potassium sorbate. In some embodiments, the excipient base comprise about 0.1%, about 0.2%, about 0.3%, about 0.4%, or about 0.5% w/v of potassium sorbate. In some embodiments, the excipient base comprises about 0.2% w/v of potassium sorbate.

One major challenge is to mask the bitter taste of Compound 1. Sweeteners are important in masking often bitter and unpleasant taste of oral solutions. Sweetener can include both natural and unnatural (including artificial and synthetic) as well as other taste masking agents and compositions. In some embodiments, the excipient base comprises a natural or an artificial sweetener, or any combination thereof.

In some embodiments the excipient base comprises a natural sweetener. Natural sweeteners include sucrose, glucose, fructose, and the like.

In some embodiments, the excipient base comprises an artificial sweetener. Artificial sweeteners include but are not limited to acesulfame potassium (Ace K), advantame, alitame, aspartame, aspartame-acesulfame, sodium cyclamate, monoammonium glycyrrihizinate, neohesperidin dihdrochalcone, neotame (NutraSweet), saccharin, stevia (steviol glycoside), sucralose, sugar alcohols or polyols. Sugar alcohols or polyols include arabitol, glycerol, sorbitol, xylitol, mannitol, erythritol and lactitol. In some embodiments, the excipient base comprises acesulfame potassium (Ace K), advantame, alitame, aspartame, aspartame-acesulfame, sodium cyclamate, monoammonium glycyrrihizinate, neohesperidin dihdrochalcone, neotame (NutraSweet), saccharin, stevia (steviol glycoside), or sucralose, or a combination thereof.

In some embodiments, the excipient base comprises acesulfame potassium, aspartame, neotame, saccharin, stevia, or sucralose, or a combination thereof. In some embodiments, the excipient base comprises acesulfame potassium. In some embodiments, the excipient base comprises aspartame. In some embodiments, the excipient base comprises neotame. In some embodiments, the excipient base comprises saccharin. In some embodiments, the excipient base comprises sucralose.

Sucralose is about 320 to 1,000 times sweeter than sucrose, three times as sweet as both aspartame and acesulfame potassium, and twice as sweet as sodium saccharin. A common brand names include Splenda. In some embodiments, the excipient base comprises sucralose. In some embodiments, the excipient base comprises from about 0.1 mg/mL to about 60.0 mg/mL, or from about 1.0 mg/mL to about 30.0 mg/mL, or any amount therein of sucralose. In some embodiments, the excipient base comprises from about 1.0 mg/mL to about 20.0 mg/mL of sucralose. In some embodiments, the excipient base comprises about 10 mg/mL, about 20 mg/mL, 30 mg/mL, about 40 mg/mL, about 50 mg/mL, or about 60 mg/mL of sucralose. In some embodiments, the excipient base comprises about 1.0 mg/mL, about 2.0 mg/mL, 3.0 mg/mL, about 4.0 mg/mL, about 5.0 mg/mL, about 6.0 mg/mL, about 7.0 mg/mL, about 8.0 mg/mL, about 9.0 mg/mL or about 10.0 mg/mL of sucralose. In some embodiments, the excipient base comprises about 1.0 mg/mL of sucralose. In some embodiments, the excipient base comprises about 5.0 mg/mL of sucralose. In some embodiments, the excipient base comprises about 10.0 mg/mL of sucralose.

In some embodiments, the excipient base comprises from about 0.01% to about 1.0% w/v of sucralose. In some embodiments, the excipient base comprises about 0.1%, about 0.2%, about 0.3%, about 0.4%, about 0.5%, about 0.6%, about 0.7%, about 0.8%, or about 0.9% w/v of sucralose.

In some embodiments, the excipient base comprises a buffer or pH modifying agent Common buffers and pH modifiers include citric acid, sodium hydroxide, potassium hydroxide, and the like. In some embodiments, the excipient base comprises citric acid.

In some embodiments, the excipient base comprises from about 0.01 mg/mL to about 10.0 mg/mL of citric acid. In some embodiments, the excipient base comprises from about 0.1 mg/mL to about 10.0 mg/mL of citric acid. In some embodiments, the excipient base comprises from about 1.0 mg/mL to about 6.0 mg/mL of citric acid. In some embodiments, the excipient base comprises about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, about 4.0 mg/mL, about 4.2 mg/mL, about 4.4 mg/mL, about 4.6 mg/mL, about 4.8 mg/mL, about 5.0 mg/mL, about 5.2 mg/mL, about 5.4 mg/mL, about 5.6 mg/mL, about 5.8 mg/mL, or about 6.0 mg/mL of citric acid. In some embodiments, the pharmaceutical composition comprises about 1.5 mg/mL of citric acid. In some embodiments, the pharmaceutical composition comprises about 2.0 mg/mL of citric acid. In some embodiments, the pharmaceutical composition comprises about 2.5 mg/mL of citric acid. In some embodiments, the pharmaceutical composition comprises about 3.0 mg/mL of citric acid. In some embodiments, the pharmaceutical composition comprises about 4.0 mg/mL of citric acid. In some embodiments, the pharmaceutical composition comprises about 5.0 mg/mL of citric acid. In some embodiments, the pharmaceutical composition comprises about 6.0 mg/mL of citric acid.

In some embodiments, the excipient base comprises from about 0.01% to about 1.0% w/v of citric acid. In some embodiments, the excipient base comprises from about 0.1% to about 0.8% w/v of citric acid. In some embodiments, the excipient base comprises about 0.1%, about 0.15%, about 0.2%, about 0.3%, about 0.4%, about 0.5%, about 0.6%, about 0.7%, about 0.8%, or about 0.9% w/v of citric acid. In some embodiments, the excipient base comprise about 0.15% w/v of citric acid. In some embodiments, the excipient base comprise about 0.2% w/v of citric acid. In some embodiments, the excipient base comprise about 0.3% w/v of citric acid. In some embodiments, the excipient base comprise about 0.4% w/v of citric acid. In some embodiments, the excipient base comprise about 0.5% w/v of citric acid.

In some embodiments, the excipient base comprises methyl paraben, potassium sorbate, sucralose, propylene glycol, and/or citric acid. In some embodiments, the excipient base does not comprise methyl paraben. In some embodiments, the excipient base does not comprise propylene glycol.

In some embodiments, the excipient base comprises potassium sorbate, sucralose, and/or citric acid. In some embodiments, the excipient base comprises from about 1.0 mg/mL to about 3.0 mg/mL potassium sorbate, from about 1.0 mg/mL to about 20.0 mg/mL sucralose, and/or from about 1.0 mg/mL to about 6.0 mg/mL of citric acid. In some embodiments, the excipient base comprises about 2.0 mg/mL potassium sorbate, about 5.0 mg/mL sucralose, and/or about 5.0 mg/mL of citric acid.

To improve flavor quality (palatability), the oral pharmaceutical composition further comprises a task masking agent. In some embodiments, the taste making agents includes flavoring or salts. In some embodiments, the pharmaceutical composition comprises a flavor. In some embodiments, the flavor is a cherry flavor, a grape flavor, a raspberry flavor, a strawberry flavor, or a tutti-fruity flavor. In some embodiments, the flavor is a cherry flavor. In some embodiments, the flavor is a grape flavor. In some embodiments, the flavor is a raspberry flavor. In some embodiments, the flavor is a strawberry flavor.

In some embodiments, the grape flavor is Sentient Grape Flavor Extract Natural Type WS. In some embodiments, the grape flavor is SN2000023802.

In some embodiments, the raspberry flavor is Sentient Natural and Artificial Raspberry Flavor. In some embodiments, the raspberry flavor is SN1000073269.

In some embodiments, the pharmaceutical composition comprises from about 1.0 mg/mL to about 15.0 mg/mL or from about 1.0 mg/mL to about 5.0 mg/mL of a flavor. In some embodiments, the pharmaceutical composition comprises about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, or about 15 mg/mL of a flavor. In some embodiments, the pharmaceutical composition comprises about 3.0 mg/mL of a flavor. In some embodiments, the pharmaceutical composition comprises about 3.5 mg/mL of a flavor. In some embodiments, the pharmaceutical composition comprises about 4.0 mg/mL of a flavor.

In some embodiments, the pharmaceutical composition comprises from about 2.0 mg/mL to about 5.0 mg/mL of a grape flavor. In some embodiments, the pharmaceutical composition comprises about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL of a grape flavor. In some embodiments, the pharmaceutical composition comprises about 3.0 mg/mL of a grape flavor. In some embodiments, the pharmaceutical composition comprises about 3.2 mg/mL of a grape flavor. In some embodiments, the pharmaceutical composition comprises about 3.4 mg/mL of a grape flavor. In some embodiments, the pharmaceutical composition comprises about 3.6 mg/mL of a grape flavor. In some embodiments, the pharmaceutical composition comprises about 3.8 mg/mL of a grape flavor.

In some embodiments, the pharmaceutical composition comprises from about 2.0 mg/mL to about 5.0 mg/mL of a raspberry flavor. In some embodiments, the pharmaceutical composition comprises about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL of a raspberry flavor. In some embodiments, the pharmaceutical composition comprises about 3.0 mg/mL of a raspberry flavor. In some embodiments, the pharmaceutical composition comprises about 3.2 mg/mL of a raspberry flavor. In some embodiments, the pharmaceutical composition comprises about 3.4 mg/mL of a raspberry flavor. In some embodiments, the pharmaceutical composition comprises about 3.6 mg/mL of a raspberry flavor. In some embodiments, the pharmaceutical composition comprises about 3.8 mg/mL of a raspberry flavor.

In some embodiments, the pharmaceutical composition further comprises a liquid carrier. In some embodiments, the liquid carrier is an aqueous solution. In some embodiments, the liquid carrier is selected from sterile water, normal saline, half normal saline, 5% dextrose in water (D5W), or ringers lactate solution (RL). In some embodiments, the liquid carrier is selected from sterile water.

The pH of the solution affects the compound stability and solubility. However, some preservatives become inactive at high pH and therefore cannot protect against microbial contamination. It was found that at a lower pH, Compound 1 is unstable. In some embodiments, pharmaceutical composition has a pH from about 3.0 to about 7.0. In some embodiments, the pharmaceutical composition has a pH from about 3.0 to about 6.0, or from about 5.5 to about 6.5. In some embodiments, pH of the pharmaceutical composition is about 5.5, about 5.6, about 5.7, about 5.8, about 5.9, about 6.0, about 6.1, about 6.2, about 6.3, about 6.4, or about 6.5. In some embodiments, pH of the pharmaceutical composition is about 5.5. In some embodiments, pH of the pharmaceutical composition is about 6.0.

In some embodiments, the pH of the pharmaceutical composition is above 5.5.

In some other embodiments, the composition comprising Compound 1, or a pharmaceutically acceptable salt, solvate, or hydrate thereof, is suitable for pediatric uses and can be taken by pediatric sickle cell anemia patients.

In some embodiments, the pharmaceutical composition comprising Compound 1, or a pharmaceutically acceptable salt, solvate, or hydrate thereof, is taken with food. In some embodiments, the pharmaceutical composition, is taken after a meal. In some embodiments, the pharmaceutical composition, is taken without food.

### Dosing

In some embodiments, the oral dosage ranges from about 0.001 to about 100 mg/kg body weight per day. In some embodiments, the oral dosage range is from about 0.01 to about 50 mg/kg body weight per day. In some embodiments, the oral dosage range is from about 0.05 to about 10 mg/kg body weight per day. Oral dosages are usually administered in one or more dosages, typically, one to three dosages per day. In some embodiments, the dose is administered once, twice, or three times a day. The exact dosage will depend upon the frequency and mode of administration, the gender, age, weight, and general health of the subject treated, the nature and severity of the condition treated and any concomitant diseases to be treated and other factors evident to those skilled in the art.

In some embodiments, Compound 1, or a pharmaceutically acceptable salt, solvate, or hydrate thereof, is administered to a subject in need thereof, at a dose of less than 6.0 mg/kg or less than about 4.0 mg/kg per body weight of the subject. In some embodiments, Compound 1 or a pharmaceutically acceptable salt, solvate, or hydrate thereof, is administered at a dose of from about 0.1 mg/kg to about 6.0 mg/kg per body weight of the subject. For example, Compound 1 or a pharmaceutically acceptable salt, solvate or hydrate thereof, is administered at a dose of from about 0.3 to about 3.0 mg/kg, or from about 0.3 to about 1.0 mg/kg per body weight of the subject. The patient may have sickle cell disease. The patient may be an adult (>18 years old) or a child (<18 years old). In some embodiments, the patient receives Compound 1, or a pharmaceutically acceptable salt, solvate, or hydrate thereof, at a dose of around 0.3 mg/kg, around 0.2 mg/kg, around 0.1 mg/kg, or around 0.05 mg/kg per body weight of the subject. In some embodiments, the patient receives Compound 1, or a pharmaceutically acceptable salt, solvate, or hydrate thereof, at about 1 mg/kg per body weight of the subject. In some embodiments, the patient receives Compound 1, or a pharmaceutically acceptable salt, solvate, or hydrate thereof, at about 3 mg/kg per body weight of the subject. In some embodiments, the patient receives Compound 1, or a pharmaceutically acceptable salt, solvate, or hydrate thereof, at about 6 mg/kg per body weight of the subject.

In some embodiments, the patient receives Compound 1, or a pharmaceutically acceptable salt, solvate, or hydrate thereof, at about 0.1 mg/kg per body weight of the subject.

In some embodiments, the patient receives Compound 1, or a pharmaceutically acceptable salt, solvate, or hydrate thereof, at about 0.3 mg/kg per body weight of the subject.

In some embodiments, the patient receives Compound 1, or a pharmaceutically acceptable salt, solvate, or hydrate thereof, at about 0.5 mg/kg per body weight of the subject.

In some embodiments, the patient receives Compound 1, or a pharmaceutically acceptable salt, solvate, or hydrate thereof, at about 1 mg/kg per body weight of the subject.

In some embodiments, the patient receives Compound 1, or a pharmaceutically acceptable salt, solvate, or hydrate thereof, at about 5 mg/kg per body weight of the subject.

In some embodiments, the patient receives Compound 1, or a pharmaceutically acceptable salt, solvate, or hydrate thereof, at about 10 mg/kg per body weight of the subject.

In some embodiments, Compound 1, or a pharmaceutically acceptable salt, solvate, or hydrate thereof is administered to a patient in need thereof, at a flat dose of about 20 mg, about 50 mg, about 100 mg, 150 mg, about 200 mg, about 300 mg, about 400, about 500 mg, or about 600 mg per day. In some embodiments, Compound 1, or a pharmaceutically acceptable salt, solvate, or hydrate thereof is administered to a patient at a dose of about 50 mg, about 100 mg, about 150 mg, about 200 mg, about 250 mg, about 300 mg, or about 350 mg. In some embodiments, Compound 1, or a pharmaceutically acceptable salt, solvate, or hydrate thereof is administered at a dose of about 50 mg. In some embodiments, Compound 1, or a pharmaceutically acceptable salt, solvate, or hydrate thereof is administered at a dose of about 100 mg. In some embodiments, Compound 1, or a pharmaceutically acceptable salt, solvate, or hydrate thereof is, administered at a dose of about 150 mg. In some embodiments, Compound 1, or a pharmaceutically acceptable salt, solvate, or hydrate thereof, is administered at a dose of about 200 mg. In some embodiments, Compound 1, or a pharmaceutically acceptable salt, solvate, or hydrate thereof, is administered at a dose of about 250 mg. In some embodiments, Compound 1, or a pharmaceutically acceptable salt, solvate, or hydrate thereof, is administered at a dose of about 300 mg. In some embodiments, Compound 1, or a pharmaceutically acceptable salt, solvate, or hydrate thereof, is administered at a dose of about 350 mg. In some embodiments, Compound 1, or a pharmaceutically acceptable salt, solvate or hydrate thereof, is administered at a dose of about 400 mg.

In some embodiments of the pharmaceutical composition, Compound 1, or a pharmaceutically acceptable salt, solvate, or hydrate thereof, is administered at a maximum dose per day or per dose. In some embodiments, a total combined dose of 1 g of Compound 1, or a pharmaceutically acceptable salt, solvate, or hydrate thereof, is administered per day or per dose. In some embodiments, a total combined dose of 600 mg Compound 1, or a pharmaceutically acceptable salt, solvate, or hydrate thereof, is administered per day or per dose. In some embodiments, a total combined dose of 500 mg Compound 1, or a pharmaceutically acceptable salt, solvate, or hydrate thereof, is administered per day or per dose. In some embodiments, a total combined dose of 400 mg Compound 1, or a pharmaceutically acceptable salt, solvate, or hydrate thereof, is administered per day or per dose. In some embodiments, a total combined dose of 300 mg Compound 1, or a pharmaceutically acceptable salt, solvate, or hydrate thereof, is administered per day or per dose. In some embodiments, a total combined dose of 200 mg Compound 1, or a pharmaceutically acceptable salt, solvate, or hydrate thereof, is administered per day or per dose. In some embodiments, Compound 1, or a pharmaceutically acceptable salt, solvate, or hydrate thereof, is administered to a patient, wherein Compound 1, or a pharmaceutically acceptable salt, solvate, or hydrate thereof is administered once a day. In some embodiments, the pharmaceutical composition is administered twice a day.

In some embodiments, Compound 1, or a pharmaceutically acceptable salt, solvate, or hydrate thereof is administered to a patient, wherein Compound 1, or a pharmaceutically acceptable salt, solvate, or hydrate thereof, is administered once a day with food. It has been found that food can dramatically reduce the adverse event profile. The incidence and severity of the side effects, such as nausea, emesis and headache, can be reduced when Compound 1, or a pharmaceutically acceptable salt, solvate, or hydrate thereof, is taken with food.

In some embodiments, Compound 1, or a pharmaceutically acceptable salt, solvate, or hydrate thereof, is administered to a patient, wherein Compound 1, or a pharmaceutically acceptable salt, solvate, or hydrate thereof is administered once a day for at least 7 days, 10 days, 2 weeks, 3 weeks, 4 weeks, 1 month, 2 months, 3 months, 4 months, 6 months, 7 months, 8 months, 9 months, 10 months, 11 months, a year, 1.5 years, or 2 years. In some embodiments, the patient is treated for 3 months. In some embodiments, the patient is treated for 6 months. In some embodiments, the patient is treated for 1 year. In some embodiments, the patient is treated for 1.5 years. In some embodiments, the patient is treated for 2 years, 3 years, 4 years, 5 years, over 5 years, or the duration of life.

In some embodiments, the pharmaceutical compositions are presented in a unit dosage form by methods known to those skilled in the art. For illustrative purposes, a typical unit dosage form for oral administration may contain from about 0.01 to about 1000 mg, from about 0.05 to about 500 mg, or from about 0.5 mg to about 200 mg.

In some embodiments, the unit dose is formulated for a pediatric patient.

### Combination Therapies

In one embodiment, the pharmaceutical composition comprising compounds of the present disclosure is used in combination with an additional active agent, such as Hydroxyurea (HU). The compounds of the present disclosure and the additional active agent may be administered simultaneously, sequentially, or at any order. The compounds of the present disclosure and the additional active agent may be administered at different dosages, with different dosing frequencies, or via different routes, whichever is suitable.

The term “administered simultaneously”, as used herein, is not specifically restricted and means that the compounds of the present disclosure and the additional active agent are substantially administered at the same time, e.g. as a mixture or in immediate subsequent sequence.

The term “administered sequentially”, as used herein, is not specifically restricted and means that the compounds of the present disclosure and the additional active agent are not administered at the same time but one after the other, or in groups, with a specific time interval between administrations. The time interval may be the same or different between the respective administrations of the compounds of the present disclosure and the additional active agent and may be selected, for example, from the range of 2 minutes to 96 hours, 1 to 7 days or one, two, or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the compounds of the present disclosure and the additional active agent is not particularly restricted. For example, when the compounds of the present disclosure and one additional active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when the compounds of the present disclosure and two or more other active agents are combined in a composition. The compounds of the present disclosure compounds of the present disclosure may comprise a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

### Methods of Using Compounds of the Disclosure

PDE9 is expressed specifically in the human haematopoietic system including neutrophils, reticulocytes erythroid and erythroleukaemic cells. Furthermore, SCD patients exhibit a marked and significant elevation of PDE9 expression in reticulocytes and neutrophils compared to healthy individuals (Almeida et al., Br J Haematol. 2008 September; 142(5), 836). Evidence additionally demonstrates a link between PDE9 and cell adhesion since pharmacologic PDE9 inhibition ameliorates the increased adhesive properties of SCD neutrophils (Miguel et al., Inflamm Res. 2011 July; 60(7), 633). The mechanism by which PDE9 inhibition decreases cell adhesion has been shown to be mediated by increased cGMP and decreased endothelial adhesion molecule expression. Importantly, in an animal model of SCD, the PDE9 inhibitor-mediated decrease in cell adhesion had the functional effect of increased cell survival. In addition to demonstrating decreased cell adhesion comparable to HU, PDE9 inhibition resulted in increased fetal non-sickled haemoglobin (HbF) production, which reduced the cellular concentration of abnormal haemoglobin (HbS) within red blood cells (RBCs) resulting in less polymerization of the abnormal haemoglobin and its associated sequelae. The importance of increasing HbF in treating SCD is evidenced by results of large studies like the Cooperative Study of Sickle Cell Disease, as well as studies in a variety of patient cohorts outside of the United States, showing that HbF is among the most important modifiers of this disease (Alsultan et al., Am J Hematol. 2013, 88(6), 531) as well as data showing that modifiers of HbF improve other hematological parameters (Akinsheye, Blood, 2011, 118(1):19). Finally, Almeida and colleagues demonstrated that treatment with HU combined with PDE9 inhibition in a mouse model of SCD leads to an additional beneficial amplification of the cGMP elevating effects of HU (Almeida et al., Blood. 2012 October; 120(14), 2879). In conclusion, PDE9 inhibition can modulate both the expression of fetal haemoglobin production as well as decrease cell adhesion, both mechanisms key for the treatment of SCD.

PDE9 inhibitors of the present disclosure and hydroxyurea (HU) act through different mechanisms. HU increases nitric oxide (NO) levels, which activate soluble guanylyl cyclase (sGC) to generate cGMP. PDE9 inhibitors of the present disclosure block the degradation of cGMP by inhibiting PDE9 enzymatic activity, thus elevating cGMP levels. In erythroid lineages, cGMP binds to protein kinase G (PKG) and signals synthesis of fetal gamma globin and ultimately production of HbF. In hematopoietic cells where PDE9 expression is high, the direct inhibition of PDE9 activity increases cGMP levels, which promotes decreased leucocyte adhesion.

One aspect of the present disclosure provides methods of using PDE9 inhibitors of the present disclosure and pharmaceutical compositions comprising PDE9 inhibitors of the present disclosure.

PDE9 inhibitors of the present disclosure may be used to treat sickle cell disease or any disease and/or symptom related to sickle cell disease, such as anemia, sickle-hemoglobin C disease (SC), vaso-occlusive crisis, attacks of pain (sickle cell crisis), splenic sequestration crisis, acute chest syndrome, aplastic crisis, hemolytic crisis, long-term pain, bacterial infections, and stroke.

In one embodiment, PDE9 inhibitors of the present disclosure are used to increase hemoglobin levels in the subject.

In another embodiment, PDE9 inhibitors of the present disclosure are used to increase cGMP levels in a cell or in the plasma of a subject, wherein the subject has sickle cell disease. The cell may be, but not limited to, red blood cells and/or white blood cells. The cGMP level may be increased by at least 50%, at least 100%, or at least 150%. In some embodiments, the cGMP level may be increased at least 2 times, 3 times, 4 times, 5 times, 10 times, 15 times, 20 times, or 25 times.

In another embodiment, PDE9 inhibitors of the present disclosure are used to increase fetal hemoglobin (HbF) positive red blood cell number in a subject, wherein the subject has sickle cell disease. The HbF positive red blood cell number is increased by at least 50%, at least 100%, or at least 150%. In some embodiments, the HbF positive red blood cell number is increased by at least 2 times, 3 times, 4 times, 5 times, 10 times, 15 times, 20 times, or 25 times.

In another embodiment, PDE9 inhibitors of the present disclosure are used to reduce sickle red blood cell percentage (% sickle RBC), stasis percentage (% stasis), total bilirubin, or total leucocyte count in a subject, wherein the subject has sickle cell disease. The % sickle RBC, % stasis, total bilirubin, total leucocyte count or spleen weight is decreased by at least 10%, 20%, 30%, 40%, 50%, 60% or 70%.

cGMP level may be measured with any suitable method in the art, such as enzyme immunoassay.

HbF positive cells, as used herein, means red blood cells with HbF. HbF positive cells may be measured from a blood sample with any suitable method in the art, such as electrophoresis and/or colorimetric methods.

Sickle red blood cells, sickled red blood cells, as used herein, means red blood cells with a crescent or sickle shape. Percent (%) sickle red blood cell may be measured from a blood sample with any suitable method in the art.

Stasis or microvascular stasis, as used herein, is serious slowing, or complete cessation, of blood or lymph flow through vessels. Percent (%) stasis is the number of static (no flow) venules divided by the number of flowing venules times 100. Percent (%) stasis may be measured with any suitable method in the art.

Total bilirubin, as used herein, means both unconjugated and conjugated bilirubin. Total bilirubin levels may be measured from a blood sample with any suitable method in the art.

Total leucocyte count or total white blood cell count, as used herein, is a blood test that measures the number of white blood cells in the body. It may be measured from a blood sample with any suitable method in the art.

Another aspect of the present disclosure provides methods of using a PDE9 inhibitor of the present disclosure in combination with at least one other active agent. They may be administered simultaneously or sequentially. They may be present as a mixture for simultaneous administration, or may each be present in separate containers for sequential administration.

The term “simultaneous administration”, as used herein, is not specifically restricted and means that the PDE9 inhibitor of the present disclosure and the at least one other active agent are substantially administered at the same time, e.g. as a mixture or in immediate subsequent sequence.

The term “sequential administration”, as used herein, is not specifically restricted and means that the PDE9 inhibitor of the present disclosure and the at least one other active agent are not administered at the same time but one after the other, or in groups, with a specific time interval between administrations. The time interval may be the same or different between the respective administrations of PDE9 inhibitor of the present disclosure and the at least one other active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the PDE9 inhibitor of the present disclosure and the at least one other active agent is not particularly restricted. For example, when a PDE9 inhibitor of the present disclosure and one other active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when a PDE9 inhibitor of the present disclosure and two or more other active agents are combined in a composition. The PDE9 inhibitor of the present disclosure may comprise a predetermined molar weight percentage from about 1% to 10%, or about 10% to about 20%, or about 20% to about 30%, or about 30% to 40%, or about 40% to about 50%, or about 50% to about 60%, or about 60% to about 70%, or about 70% to about 80%, or about 80% to about 90%, or about 90% to about 99% of the composition.

The other active agent may be a different PDE9 inhibitor of the present disclosure or HU. The other active agent may also be an antibiotic agent such as penicillin, a nonsteroidal anti-inflammatory drug (NSAIDS) such as diclofenac or naproxen, a pain relief medication such as opioid, or folic acid.

Yet another aspect of the present disclosure provides methods of using a PDE9 inhibitor of the present disclosure in combination with at least one other therapy, such as but not limited to blood transfusion, bone marrow transplant, or gene therapy.

### Kits and Devices

The disclosure provides a variety of kits and devices for conveniently and/or effectively carrying out methods of the present disclosure. Typically, kits will comprise sufficient amounts and/or numbers of components to allow a user to perform multiple treatments of a subject(s) and/or to perform multiple experiments.

In one embodiment, the present disclosure provides kits for treating sickle cell disease, comprising a PDE9 inhibitor compound of the present disclosure or a combination of PDE9 inhibitor compounds of the present disclosure, optionally in combination with any other active agents, such as HU, an antibiotic agent such as penicillin, a nonsteroidal anti-inflammatory drug (NSAIDS) such as diclofenac or naproxen, a pain relief medication such as opioid, or folic acid.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of PDE9 inhibitor compounds in the buffer solution over a period of time and/or under a variety of conditions.

The present disclosure provides for devices that may incorporate PDE9 inhibitor compounds of the present disclosure. These devices contain in a stable pharmaceutical formulation available to be immediately delivered to a subject in need thereof, such as a human patient with sickle cell disease.

Non-limiting examples of the devices include a pump, a catheter, a needle, a transdermal patch, a pressurized olfactory delivery device, iontophoresis devices, multi-layered microfluidic devices. The devices may be employed to deliver PDE9 inhibitor compounds of the present disclosure according to single, multi- or split-dosing regiments. The devices may be employed to deliver PDE9 inhibitor compounds of the present disclosure across biological tissue, intradermal, subcutaneously, or intramuscularly. More examples of devices suitable for delivering PDE9 inhibitor compounds include but not limited to a medical device for intravesical drug delivery disclosed in International Publication WO 2014036555, a glass bottle made of type I glass disclosed in US Publication No. 20080108697, a drug-eluting device comprising a film made of a degradable polymer and an active agent as disclosed in US Publication No. 20140308336, an infusion device having an injection micro-pump, or a container containing a pharmaceutically stable preparation of an active agent as disclosed in U.S. Pat. No. 5,716,988, an implantable device comprising a reservoir and a channeled member in fluid communication with the reservoir as disclosed in International Publication WO 2015023557, a hollow-fiber-based biocompatible drug delivery device with one or more layers as disclosed in US Publication No. 20090220612, an implantable device for drug delivery including an elongated, flexible device having a housing defining a reservoir that contains a drug in solid or semi-solid form as disclosed in International Publication WO 2013170069, a bioresorbable implant device disclosed in U.S. Pat. No. 7,326,421, contents of each of which are incorporated herein by reference in their entirety.

### Definitions

The articles “a” and “an,” as used herein, should be understood to mean “at least one,” unless clearly indicated to the contrary.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements.

In general, the term “or” as used herein shall only be interpreted as indicating exclusive alternatives (i.e. “one or the other but not both”) when preceded by terms of exclusivity, such as “either,” “one of” “only one of,” or “exactly one of” “Consisting essentially of,” when used in the claims, shall have its ordinary meaning as used in the field of patent law.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, all transitional phrases such as “comprising,” “including,” “carrying,” “having,” “containing,” “involving,” “holding,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures.

As used herein, a “subject” or a “patient” refers to any mammal (e.g., a human), such as a mammal that may be susceptible to a disease or disorder, for example, tumorigenesis or cancer. Examples include a human, a non-human primate, a cow, a horse, a pig, a sheep, a goat, a dog, a cat, or a rodent such as a mouse, a rat, a hamster, or a guinea pig. In various embodiments, a subject refers to one that has been or will be the object of treatment, observation, or experiment. For example, a subject can be a subject diagnosed with cancer or otherwise known to have cancer or one selected for treatment, observation, or experiment on the basis of a known cancer in the subject.

As used herein, “treatment” or “treating” refers to amelioration of a disease or disorder, or at least one sign or symptom thereof “Treatment” or “treating” can refer to reducing the progression of a disease or disorder, as determined by, e.g., stabilization of at least one sign or symptom or a reduction in the rate of progression as determined by a reduction in the rate of progression of at least one sign or symptom. In another embodiment, “treatment” or “treating” refers to delaying the onset of a disease or disorder.

As used herein, “prevention” or “preventing” refers to a reduction of the risk of acquiring or having a sign or symptom a given disease or disorder, i.e., prophylactic treatment.

The phrase “therapeutically effective amount” as used herein means that amount of a compound, material, or composition comprising a compound of the present teachings that is effective for producing a desired therapeutic effect. Accordingly, a therapeutically effective amount treats or prevents a disease or a disorder, e.g., ameliorates at least one sign or symptom of the disorder. In various embodiments, the disease or disorder is a cancer.

A dash (“-”) that is not between two letters or symbols is used to indicate a point of attachment for a substituent. For example, —CONH₂ is attached through the carbon atom (C).

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

All numerical ranges herein include all numerical values and ranges of all numerical values within the recited range of numerical values. As a non-limiting example, (C₁-C₆) alkyls also include any one of C₁, C₂, C₃, C₄, C₅, C₆, (C₁-C₂), (C₁-C₃), (C₁-C₄), (C₁-C₅), (C₂-C₃), (C₂-C₄), (C₂-C₅), (C₂-C₆), (C₃-C₄), (C₃-C₅), (C₃-C₆), (C₄-C₅), (C₄-C₆), and (C₅-C₆) alkyls.

Further, while the numerical ranges and parameters setting forth the broad scope of the disclosure are approximations as discussed above, the numerical values set forth in the Examples section are reported as precisely as possible. It should be understood, however, that such numerical values inherently contain certain errors resulting from the measurement equipment and/or measurement technique.

### List of Abbreviations and Terms

¹H-NMR: Proton Nuclear Magnetic Resonance spectroscopy

### ADME: Absorption, Distribution, Metabolism, and Excretion

AE: Adverse event
AUC₀₋₂₄: area under the concentration-time curve from time 0 to 24 hours postdose
BBB: blood-brain barrier
Cₘₐₓ: maximum plasma concentration
cGMP: cyclic guanosine monophosphate
DMSO: dimethyl sulfoxide
DSFC: dorsal skin-fold chambers
F cells: blood cells with fetal hemoglobin
FIH: first in human
FTIR: Fourier transform infrared spectroscopy
GC: gas chromatography
HBB: hemoglobin subunit beta
HbF: fetal hemoglobin
HBG: gamma-globin gene
HbS: sickle hemoglobin
hERG: human ether-à-go-go related gene
HPLC: high-performance liquid chromatography
HU: hydroxyurea
IC: inhibitory concentration
IC₅₀: a half minimal inhibitory concentration
ICAM-1: intercellular adhesion molecule-1

### ICH: International Conference on Harmonization

ICP-MS: inductively coupled plasma mass spectroscopy
IV: intravenous
MAD: multiple-ascending dose
MTD: maximum tolerated dose
NO: nitric oxide
NOAEL: no-observed-adverse-effect level
PD: pharmacodynamic
PDE9: phosphodiester-9
PEG polyethylene glycol
PIC: Powder in capsule
PK: pharmacokinetic(s)
PKG: protein kinase G
RBC: red blood cell
RH: relative humidity
SCD: sickle cell disease
SD: standard deviation
SEM: standard error of the mean
sGC: soluble guanylyl cyclase
t½: half-life

### TK: Toxicokinetic

Tₘₐₓ: time of maximum concentration
VOC: vaso-occlusive crisis
WBC: white blood cell
w/w %: weight/weight percent

### EXAMPLES

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety.

### Example 1. Synthesis and Formulation of Compound 1

Compound 1 is an enantiomer of 6-[4-methyl-1-(pyrimidin-2-ylmethyl)pyrrolidin-3-yl]-3-tetrahydropyran-4-yl-7H-imidazo[1,5-a]pyrazin-8-one disclosed in WO 2013/053690. Compound 1 may be prepared from chiral-selective purification from 6-[4-methyl-1-(pyrimidin-2-ylmethyl)pyrrolidin-3-yl]-3-tetrahydropyran-4-yl-7H-imidazo[1,5-a]pyrazin-8-one prepared according to the method disclosed in WO 2013/053690, the contents of which are incorporated herein by reference in their entirety. Compound 1 may also be prepared with the method disclosed in WO 2017/005786, the contents of which are incorporated herein by reference in their entirety.

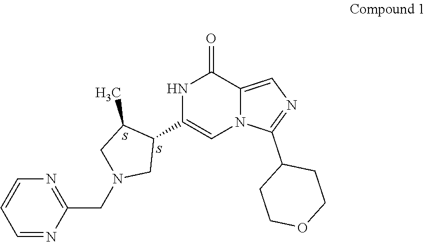

Compound 1 drug product to be used in ongoing clinical development is an immediate release tablet. The coating is may be used to assure uniformity of appearance across different tablet strengths and with the placebo.

Earlier clinical studies were performed with Compound 1 drug substance directly filled into opaque white gelatin capsules (Powder in Capsule, PIC) with no excipients or processing aids. An excipient-blended tablet form of the drug product for oral administration has been developed, as this allowed for scale-up of the manufacturing process and assurance of content uniformity. These tablets were tested for defined limits for purity, potency, dissolution, total aerobic microbial count, as well as total yeast and mold count. In addition, tests for specified microorganisms were performed.

Each tablet comprises 20 mg, 50 mg, 100 mg, 150 mg, or 200 mg of Compound 1 drug substance (the monohydrate of the API) or placebo. A representative tablet composition is shown below in Table 1.

TABLE 1
Compound 1 50 mg coated tablets

| Component | Weight/Unit (mg) |
|---|---|
| Tablet Blend | |
| Compound 1 Drug Substance | 50.0 |
| Microcrystalline Cellulose | 318.0 |
| Pre-gelatinized Starch | 20.0 |
| Colloidal Silicon Dioxide | 8.0 |
| Magnesium Stearate | 4.0 |
| Tablet Core Total | 400.0 |
| Coating Solution | |
| Opadry II White Film Coating | 40.0 |
| Purified Water | — |
| Final | 440.0 |

Purified water is removed during processing.

All tablets were configured such that the target weight of the core tablets was 400 mg, and the target weight of the coated tablet was 440 mg. To accomplish this, the amounts of Compound 1 and Microcrystalline Cellulose were adjusted accordingly. All other excipient amounts remained constant.

### Example 2. Compound 1 Reduces White Cell Adhesion and Activation

Polymorphic mononuclear cells (PMN), particularly neutrophils, play a critical role in pathogenesis of sickle cell disease (SCD) and activated neutrophils have been shown to be more adhesive to each other, platelets and the vascular endothelium. Recently several drugs targeting white cell binding to endothelial cells, have been advancing in clinical studies in patients. Compound 1 is able to increase expression of fetal hemoglobin in patient derived cells and murine models of SCD and reduce vessel occlusion in SCD murine models. In this Example, the ability of Compound 1 to reduce the adhesive properties of neutrophils from SCD patients and reduce sE-Selectin (sE-Sel) and markers of PMN activation in murine SCD models was studied.

Endothelial E-selectin (E-Sel) slows leukocyte rolling, which is followed by stationary adhesion and transmigration of activated leukocytes. Plasma levels of sE-Sel, produced by the enzymatic cleavage of the extracellular domains of E-Sel, are increased in SCD patients and this may be mediated by its interaction with leukocytes. In the Townes mouse model, plasma sE-Sel is increased 144% (139 mg/ml) over levels seen in control mice (57 mg/ml). This was reduced significantly in Townes mice treated with Compound 1, where plasma sE-Sel levels were elevated by only 61% over control mice (92 mg/ml).

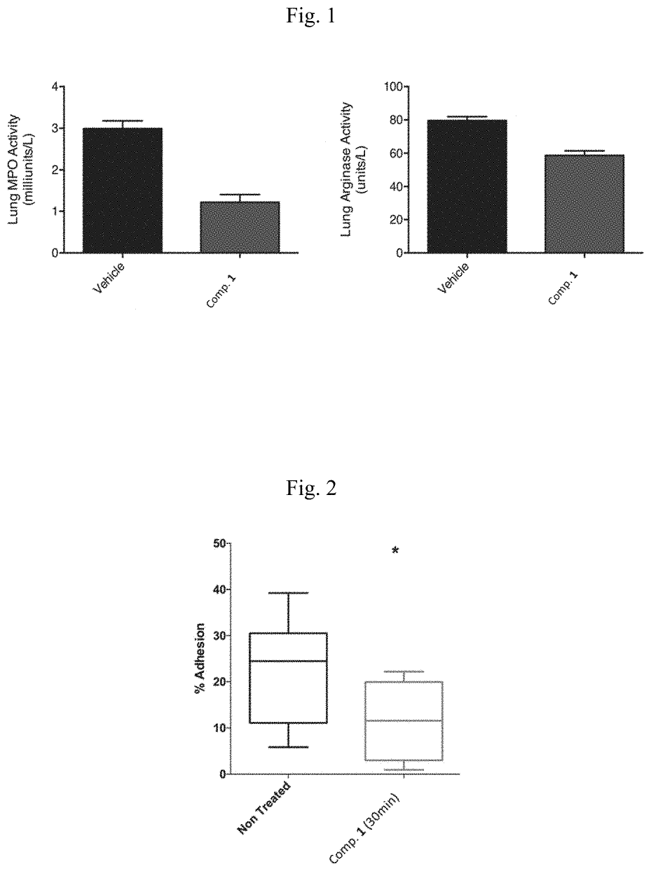

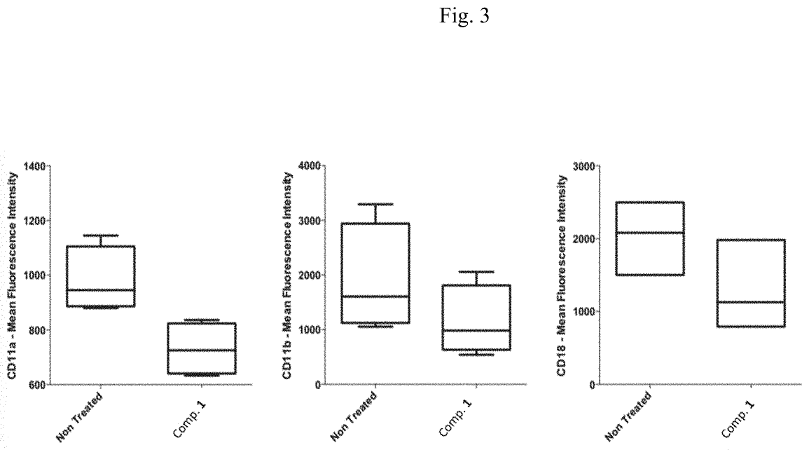

It was found that Compound 1 reduced circulating levels of PMNs in SCD models, but not in long term studies in healthy animals. This appears to be accompanied by a Compound 1-mediated reduction in disease specific cell activation including 67% lower levels of myeloid derived myeloperoxidase (MPO) and 26% lower levels of neutrophil derived arginase in the lung (FIG. 1). Using a previously described in vitro adhesion assay mimicking blood flow, where activated endothelial cells HMEC-1 line the inner surface of microchannels, perfused whole blood samples from SS (the most common form of sickle cell) patients showed that neutrophils aggregate and bind to the endothelial monolayer. This was quantified by real time monitoring of the green florescent patches in the microchannel, as neutrophils are labeled by a specific Alexa Fluor® 488-conjugated antibody before the perfusion step. Untreated, patient neutrophils showed a significant amount of adhesion to activated HMEC-1. When added to blood samples prior to the perfusion step, Compound 1 reduced adhesions significantly and in a dose dependent manner. The inhibitory effect was initiated as early as 15 min of incubation, with the most potent inhibition of adhesion observed for 30 min incubation with 30 μM of Compound 1. Under these conditions, adhesion was reduced an average of 54% (p=0.03) (FIG. 2). Mechanistically, not willing to be bound by any theory, Compound 1 may target the stationary adhesion step of neutrophils as it lowered expression levels of key neutrophil integrins including CD11a [reduced 23% (p=0.002)], CD11b [reduced 39% (ns)) and CD18 [reduced 47% (p=0.03)) (FIG. 3).

Together, these data indicate a role for Compound 1 in reducing PBMCs mediated pathology in SCD by targeting the abnormal adhesion of neutrophils independently from their cell count in the circulation.

### Example 3. Effects of Compound 1 vs. Hydroxyurea (HU)

A series of studies were conducted to compare the effects of Compound 1 against hydroxyurea (HU). A summary of the results is shown in the table below in Table 2.

TABLE 2
Compound 1 versus hydroxyurea (HU)

| Model System | Data with Compound 1 |
|---|---|
| Erythroid cell lines | Increased cGMP levels vs. HU; |
| (K562, UT-7) | induction of HbF (RNA, protein, etc.) |
| SCD patient derived cell lines | F-cells % increase; increases in HbF |
| In-vivo SCD: Townes | Statistically better results than HU |
| (RBC/WBC) | across most RBC/WBC parameters |
| In-vivo SCD: vaso occlusion | Better results vs. HU; combo |
| (WBC) | Compound 1 + HU |
| | shows early signs of synergism |
| In-vivo cognition: C57B1/6J | No CNS activity; no change in |
| | locomotor activity or fear |
| | conditioning (other PDE9i |
| | showed changes) |

The effects of Compound 1 is compared with HU in in vitro studies in erythroid cell lines (K562 and UT-7). Compound 1 shows increased cGMP levels compared with HU and shows an induction of HbF (RNA, protein, etc.). In a study performed in SCD patient derived cell lines, Compound 1 treated cells showed F-cells % increase and increases in HbF.

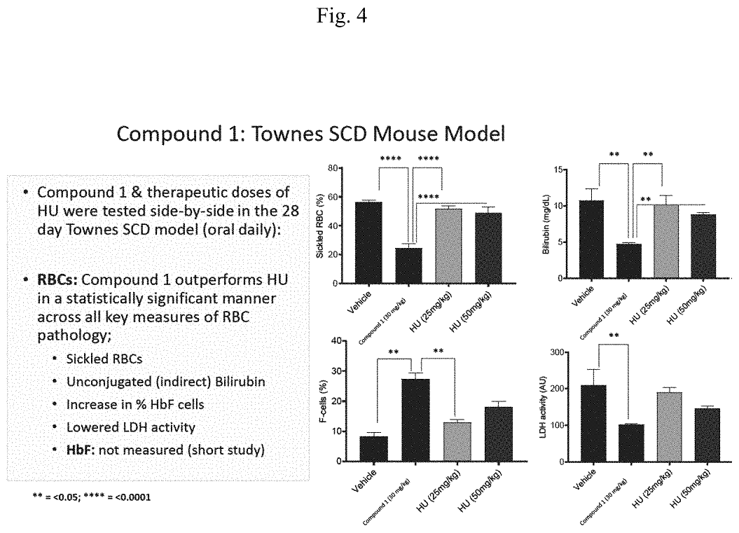

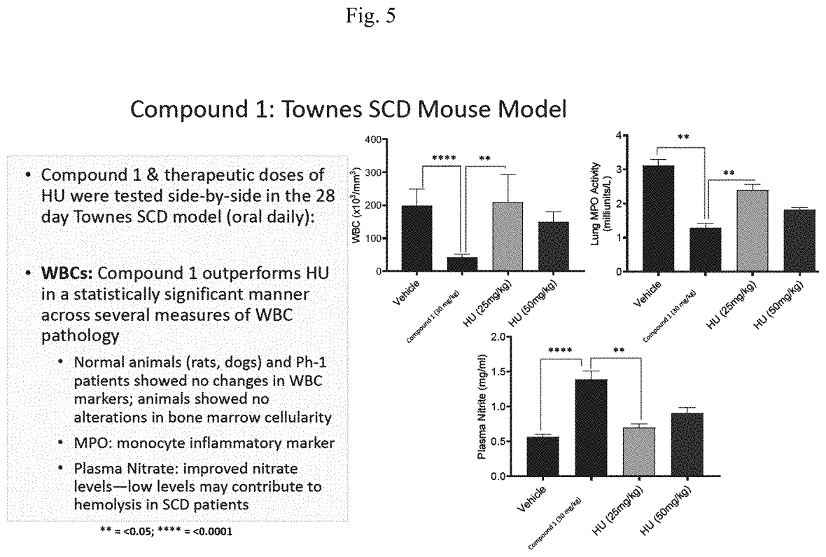

In one study, Compound 1 and therapeutic doses of HU (25 mg/kg and 50 mg/kg) were tested side-by-side in the 28 day Townes SCD model (oral daily). As shown in FIG. 4, Compound 1 outperforms HU in a statistically significant manner across all key measures of red blood cell (RBC) pathology including sickled RBCs; unconjugated (indirect) bilirubin; increase in % HbF cells; and lowered lactate dehydrogenase (LDH) activity. As shown in FIG. 5, Compound 1 also outperforms HU in a statistically significant manner across several measures of WBC (white blood cell) pathology. MPO is a monocyte inflammatory marker. Plasma nitrate is improved nitrate levels. Lower levels of plasma nitrate may contribute to hemolysis in SCD patients.

### Example 4. A Phase 2a, Randomized, Double-Blind, Placebo-Controlled Study of Compound 1 in Adult Patients with Sickle Cell Anemia (SCA)

### Objectives:

Primary Objectives: To assess the safety and tolerability of Compound 1 in adult patients with sickle cell anemia (SCA), defined as homozygous sickle hemoglobin (HbSS) or sickle-β⁰ thalassemia, who are not receiving hydroxyurea (HU) and in adult SCA patients who are receiving a stable dose of HU.

Secondary Objectives: To characterize the pharmacokinetic (PK) profile of Compound 1 in adult patients with SCA who are/are not receiving a stable dose of HU; to characterize the PK profile of HU in adult patients with SCA before and after receiving Compound 1 to determine if there is a clinically relevant PK interaction.

Exploratory Objectives: To assess the pharmacodynamic (PD) effects of Compound 1 in adult patients with SCA who are/are not receiving stable HU; to assess the potential efficacy of Compound 1 on SCA-related clinical outcome measures in adult patients with SCA who are/are not receiving stable HU.

### Methodology:

This is a randomized, double-blind, placebo-controlled study to evaluate the safety, tolerability, PK, and exploratory PD and clinical outcomes of the phosphodiesterase 9 (PDE9) inhibitor, Compound 1, administered once daily for 16 to 24 weeks in 2 populations of patients with SCA: those who are not receiving HU (Population A) and those who are currently receiving a stable dose of HU according to standard of care (Population B). Up to approximately 36 patients are enrolled in Population A and 18 patients are enrolled in Population B.

Population A: Following a Screening period of up to 4 weeks, eligible patients in Population A (i.e., those not receiving HU) receive either Compound 1 or placebo for a total of 24 weeks. On Day 1, patients are randomized 1:1:1 to receive oral Compound 1 30 mg, 50 mg, Compound 1 100 mg, or placebo daily for the first 12 weeks; for the second 12 weeks (Weeks 13-24), each patient's dose may be doubled (i.e., from 50 mg to 100 mg; from 100 mg to 200 mg; or placebo). (Note because placebo and all dose levels of Compound 1 are the same in appearance, dose escalation does not affect study medication blinding). Throughout the study, all available clinical data are reviewed approximately every 2 weeks, and dose escalation occurs on an individual patient basis on Day 85 only if approved based upon review of each patient's individual clinical safety data.

Population B: Following a Screening period of up to 4 weeks, eligible patients in Population B (i.e., those receiving stable HU) enter a lead-in period and have blood samples drawn to characterize the PK profile of the patient's prescribed dose of HU in the absence of Compound 1 (i.e., to characterize the patient's baseline HU PK profile). Two full baseline HU PK profiles (with blood samples drawn over a 10-hour period at least 48 hours apart) are determined.

Compound 1 dosing in Population B do not begin until at least 4 weeks of safety data from 6 patients in Population A have been reviewed and determined that it is safe and appropriate to begin dosing in Population B. Following approval to initiate dosing in Population B and once the baseline HU PK blood draws are complete, patients are randomized 2:1 on Day 1 to receive oral Compound 1 30 mg, 50 mg or placebo for 16 weeks. For the first 4 weeks (Weeks 1-4), patients receive study medication according to their randomized treatment assignment; for the following 12 weeks (Weeks 5-16), each patient's dose may be doubled (e.g., from 50 mg to 100 mg; or placebo). As in Population A, dose escalation occurs on Day 29 only if approved based upon review of each patient's individual clinical safety data.

### Study Design Rationale:

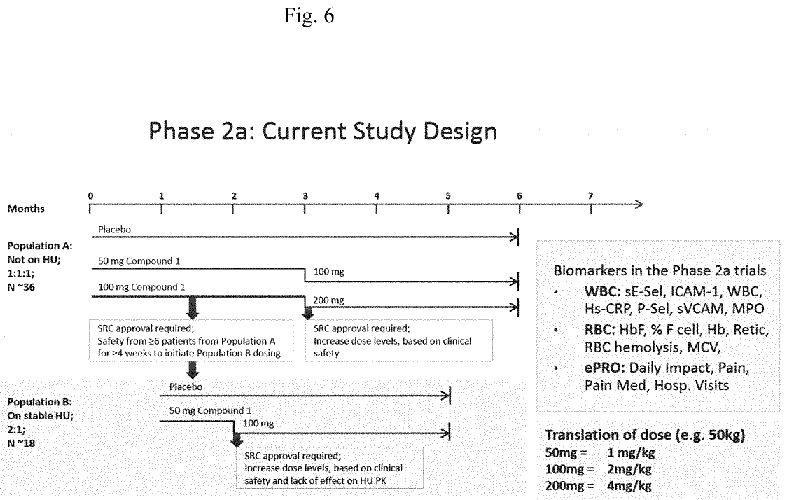

A summary of the study design is shown in FIG. 6. This is the first study in a patient population (patients with SCA), and as such, is designed to examine the safety, tolerability, and PK, as well as the potential PD effects and clinical efficacy, of Compound 1 across a range of doses in adult patients with SCA. Given the possibility that Compound 1, if approved, could be administered as a single agent or co-administered with HU, the effects of Compound 1 are evaluated in SCA patients who are not receiving HU or any other treatment known to modulate HbF levels (Population A) as well as in those who are currently receiving a stable dose of HU (Population B).

Available nonclinical and healthy volunteer clinical data suggest that Compound 1 is safe and well tolerated at once daily doses of 30, 50, 100, and 200 mg and that a potentially clinical beneficial PD effect is likely to be observed when a dose of at least 100 mg is administered for at least 24 weeks. Therefore, Population A is designed to explore the PD dose response in patients as well as the tolerability of the 200 mg dose level in sickle cell patients who have tolerated the 100 mg dose well.

Results from Population B are intended to provide information on Compound 1 when administered concomitantly with HU, both of which increase HbF levels through alternative biochemical pathways that increase intracellular cGMP. Because there are no clinical data to support administration of Compound 1 concomitantly with HU, patients in Population B initiate Compound 1 dosing at the low dose (30 mg or 50 mg) used in Population A and only escalate to the 100 mg dose if the 50 mg dose has been safe and tolerated for 4 weeks. In addition, although available nonclinical data do not suggest that concomitant administration of HU with Compound 1 would increase Compound 1 exposure, dosing in Population B does not initiate until 4 weeks of safety data are available from Population A in 2 patients each at 30 mg or 50 mg (starting dose in Population B) and at 100 mg (2× the starting dose) as well as placebo.

### Diagnosis and Main Criteria for Inclusion

Inclusion Criteria: Each patient must meet all of the following criteria to be enrolled in the study: 1. Male or female ≥18 or ≤50 years of age. 2. Confirmed diagnosis of SCA (HbSS or sickle-β0 thalassemia). Note, if not already documented in the patient's record, the diagnosis of SCA must be confirmed via electrophoresis, HPLC, and/or genotyping. 3. Use of HU: For patients in the Population A: Have not received HU within 90 days prior to Screening and are not planning to take HU within the next 6 months. For patients in Population B: Have received HU for at least 6 months, have been on a stable dose for at least 60 days prior to Screening, and are not planning to change the dose level, dose regimen, or discontinue HU within the next 6 months. 4. Female patients must not be pregnant and be highly unlikely to become pregnant. Male patients must be unlikely to impregnate a partner.

Exclusion Criteria: Patients who meet any of the following criteria are excluded from the study: 1. Total Hb at Screening >11.0 g/dL or <6 g/dL. 2. Reticulocyte count <100×109/L. 3. >3 hospitalizations (for at least 24 hours) for vaso-occlusive crises (VOC), including acute chest syndrome (ACS) and priapism, within the prior year. 4. Receiving chronic outpatient opioid treatment (equivalent to ≥10 mg oral morphine daily) for any reason other than avascular necrosis (AVN). Note: chronic treatment is defined as continuous daily opioid use for ≥8 weeks. 5. Blood transfusion or donation of blood or any blood product within 60 days of Day 1 or on chronic transfusion therapy regimen. 6. Positive for human immunodeficiency virus (HIV), hepatitis C (HCV) antibodies (unless the patient has successfully completed drug therapy that results in cure/clearance of HCV), and hepatitis B surface antigen (HBsAg). 7. For female patients of childbearing potential, a positive serum human chorionic gonadotropin (hCG) test (Screening) or a positive urine hCG test on Day 1. 8. Estimated glomerular filtration rate (eGFR) <50 mL/min as calculated by the equation from the Modification of Diet in Renal Disease (MDRD) Study using creatinine, age, sex, and ethnicity. 9. Alanine aminotransferase (ALT) or aspartate aminotransferase (AST) >3× the upper limit of normal (ULN). 10. Body Mass Index (BMI)<17.5 or >35 kg/m2; a total body weight <50 kg. 11. Use of PDES inhibitors (including but not limited to sildenafil, tadalafil, vardenafil) within 7 days prior to the first dose of study drug, or planning to use any time during study. 12. A history of drug or alcohol abuse as judged by the investigator within the past 1 year, or a positive alcohol (breathalyzer) test (Screening or Day −1). 13. A cancer that has not been in complete remission for at least 5 years. Patients with squamous cell or basal cell carcinoma of the skin, localized cervical cancer, or localized prostate cancer are eligible if, in the opinion of the investigator, the condition has been adequately diagnosed, and is determined to be clinically in remission, and the patient's participation in the study would not represent a safety concern. 14. A history of a clinically significant allergic reaction or hypersensitivity, as judged by the investigator, to any drug or any component of the study drug formulations used in the study. 15. On ECG, a corrected QT interval, Fridericia's formula (QTcF) >450 ms in men and >470 ms in women or the presence of clinically significant abnormalities as determined by the investigator. 16. A history of major surgery within 4 weeks or minor surgery within 2 weeks of Day 1. 17. Any flu-like syndrome or other respiratory infection within 2 weeks of Day 1 or vaccination with attenuated live virus within 4 weeks of Day 1. 18. Participation in an investigational drug or device study within 30 days prior to Day 1. 19. Use within 30 days prior to Day 1, or planning to use during the study, of any drugs or substances that are known to strongly inhibit or induce cytochrome P450 enzymes (CYPs), including but not limited to cimetidine, cyclosporine, erythromycin, omeprazole, rifampin, ritonavir, and St. John's wort. If there is any question as to whether a substance is permitted, please review the product labelling (if applicable) and consult the Sponsor. 20. Consumption of grapefruit, grapefruit juice, or grapefruit products within 24 hours prior to Day 1 or planning to consume grapefruit products during the study. 21. Use within 30 days prior to Day 1, or planning to use during the study, of any CYP3A sensitive substrates, (excluding opioids), including but not limited to alfentanil, avanafil, budesonide, buspirone, conivaptan, darifenacin, darunavir, dasatinib, dronedarone, ebastine, eletriptan, eplerenone, everolimus, felodipine, ibrutinib, indinavir, lomitapide, lurasidone, maraviroc, midazolam, naloxegol, nisoldipine, quetiapine, saquinavir, sirolimus, tacrolimus, ticagrelor, tipranavir, tolvaptan, triazolam. 22. Use within 30 days prior to Day 1, or planning to use during the study, of any drugs or substances known to be significant substrates or inhibitors of P-glycoprotein (P-gp), including but not limited to cyclosporine, lovastatin, propranolol, quinidine, and simvastatin. If there is any question as to whether a substance is permitted, please review the product labelling (if applicable) and consult the Sponsor. 23. Other prior or ongoing medical condition, physical findings, or laboratory abnormality that, in the investigator's opinion, could adversely affect the safety of the patient, make it unlikely that the course of treatment or follow-up would be completed, or impair the assessment of study results.

### Investigational Product, Dosage and Mode of Administration

Compound 1 is supplied as 50, 100 or 200 mg white tablets and is administered orally with food. The different doses of Compound 1 are visually identical in tablet form.

### Reference Therapy, Dosage and Mode of Administration

Placebo consists of tablets containing matrix absent Compound 1 and is identical in appearance to the Compound 1 tablets. Placebo is administered orally with food.

### Duration of Treatment

The total duration of the study is approximately 32 weeks for Population A, including a Screening period of up to 4 weeks, a treatment period of 24 weeks, and a 4-week follow-up assessment after the last dose of study drug is administered.

The total duration of the study is approximately 32 weeks for Population B, including a Screening period of up to 4 weeks, a lead-in period of approximately 8 weeks, a treatment period of 16 weeks, and a 4-week follow-up assessment after the last dose of study drug is administered.

### Endpoints

The endpoints for Populations A and B are the same except where noted otherwise.

Primary Endpoints: Compound 1 safety and tolerability as measured by: Incidence and severity of adverse events (AEs) and serious adverse events (SAEs); Change from baseline in 12-lead electrocardiogram (ECG) parameters, clinical laboratory tests (chemistry, hematology, coagulation, urine), and vital signs; Physical examination findings.

Secondary Endpoints: The plasma PK profile of Compound 1 after oral administration to adult patients with SCA (Populations A and B); The plasma PK profile of HU before and after oral administration of Compound 1 to adult patients with SCA (Population B only).

Exploratory Endpoints: Compound 1 PD as measured by the following (additional exploratory biomarkers may also be tested): Total hemoglobin (Hb) levels; HbF value (%); % F cells; Indices of red cell hemolysis (unconjugated bilirubin, reticulocyte count, lactase dehydrogenase [LDH], and haptoglobin levels); Soluble E-selectin (sE-Sel), Soluble P-selectin (sP-Sel) and soluble intercellular adhesion molecule 1 (sICAM-1); High sensitivity-C reactive protein (hs-CRP). Compound 1 clinical outcomes as measured by pain-related measures (frequency, severity, and duration of pain; impact of pain/fatigue on work/school and on activities of daily living; need for/use of pain medication; SCA-related events requiring professional medical or health care, including events requiring hospitalization or therapies, such as transfusions) and in the physical, social, and emotional impact of SCA as measured by the Adult Sickle Cell Quality-of-Life Measurement Information System (ASCQ-Me).

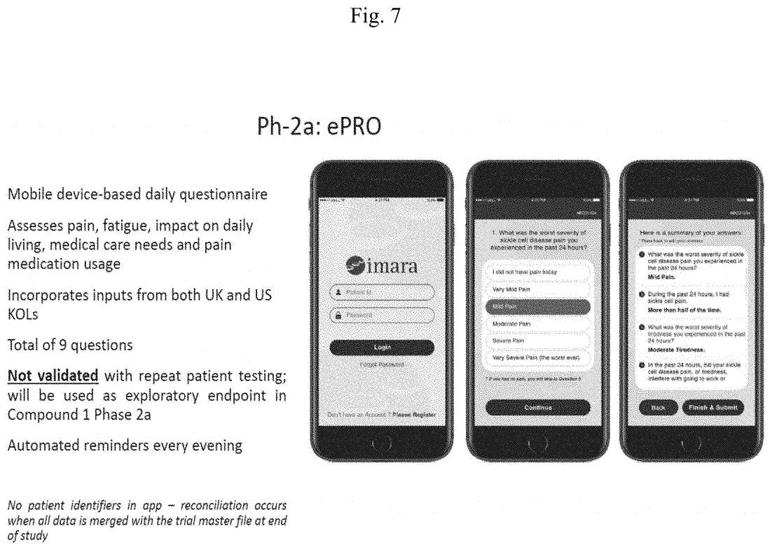

Patients receives a mobile device-based daily questionnaire which accesses pain, fatigue, impact on daily living, medical care needs, and pain medication usage. A representative sampling of screenshots of the questionnaire app are shown in FIG. 7. The questionnaire app incorporates inputs from Key Opinion Leaders (KOLs) of both United Kingdom (UK) and United States (US) and sends automated reminders every day (e.g., every evening). The questionnaire is not validated with repeat patient testing and is used as exploratory endpoint in this study.

In addition, a separate blood sample is collected for confirmation of diagnosis by electrophoresis, high performance liquid chromatography (HPLC) and/or DNA sequencing (as needed) as well as for possible pharmacogenomic analyses of genes that may affect treatment response (including but not limited to alpha globin and BCL11A).

### Example 5. Formulation Development of an Oral Solution of Compound 1

List of materials used is described below.

| No. | Material Description |
|---|---|
| 1 | Compound 1 (API; Drug Substance) |
| 2 | Methyl paraben |
| 3 | Potassium sorbate |
| 4 | Sucralose |
| 5 | Strawberry dry flavor |
| 6 | Propylene Glycol |
| 7 | Citric acid anhydrous powder |

In this study, a liquid oral formulation of Compound 1 that can be used for pediatric patients was developed. The properties of solution were characterized, including appearance, % assay, organic impurity and pH.

One challenge was that Compound 1 has an extremely bitter tasting that is difficult to mask. There is no obvious excipient that can mask its taste. Another major challenge was to optimize the excipients quantity. It was found that at a lower pH, Compound 1 is unstable. In some embodiments, a pharmaceutical composition of pH above 5.5 comprising Compound 1 is provided. However, the preservatives become inactive when the pH is too high and cannot protect against microbial contamination. Extensive formulation development and manufacturing process studies were carried out. Other challenges are the excipient compatibility and stability of the API in solution.

Formulation Development

I. Excipient Compatibility Study

The excipient compatibility was evaluated in 5 prototypes. The testing parameters were physical appearance, pH, assay and total related substances (TRS). All these samples were further tested for taste testing. The excipient compatibility study compositions are listed in below in Table 3.

TABLE 3
Excipient compatibility study design for impurities and
assay test in prototypes.

| Sample | Components | Quantity (mg)/mL | % w/v |
|---|---|---|---|
| Prototype 1 | Drug (API) | 10 | 1.0 |
| | Methyl paraben | 2 | 0.2 |
| | Sodium Benzoate | 2 | 0.2 |
| | Sucralose | 40 | 4.0 |
| | Strawberry Flavor | 10 | 1.0 |
| | Citric acid | 10 | 1.0 |
| | Propylene Glycol | 150 | 15.0 |
| | Purified water | Quantity Sufficient to | ~77.6% |
| | | produce 1 mL | |
| Prototype 2 | Drug (API) | 10 | 1.0 |
| | Methyl paraben | 2 | 0.2 |
| | Sodium Benzoate | 2 | 0.2 |
| | Sucralose | 40 | 4.0 |
| | Cherry dry flavor | 10 | 1.0 |
| | Citric acid | 10 | 1.0 |
| | Propylene Glycol | 150 | 15.0 |
| | Purified water | Quantity Sufficient to | ~77.6% |
| | | produce 1 mL | |
| Prototype 3 | Drug (API) | 10 | 1.0 |
| | Methyl paraben | 2 | 0.2 |
| | Sodium Benzoate | 2 | 0.2 |
| | Sucralose | 40 | 4.0 |
| | Tutti-fruity dry flavor | 10 | 1.0 |
| | Citric acid | 10 | 1.0 |
| | Propylene Glycol | 150 | 15.0 |
| | Purified water | Quantity Sufficient to | ~77.6% |
| | | produce 1 mL | |
| Prototype 4 | Drug (API) | 10 | 1.0 |
| | Sucralose | 40 | 4.0 |
| | Cherry dry flavor | 10 | 1.0 |
| | Citric acid | 10 | 1.0 |
| | Propylene Glycol | 150 | 15.0 |
| | Purified water | Quantity Sufficient to | ~78.0% |
| | | produce 1 mL | |
| Prototype 5 | Drug (API) | 10 | 1.0 |
| | Methyl paraben | 2 | 0.2 |
| | Sodium Benzoate | 2 | 0.2 |
| | Sucralose | 40 | 4.0 |
| | Cherry dry flavor | 10 | 1.0 |
| | Citric acid | 10 | 1.0 |
| | Purified water | Quantity Sufficient to | ~92.6% |
| | | produce 1 mL | |

Per taste testing results, bitterness could be detected for all these samples. Compound 1 has a solubility of ˜1 g/ml in water. At this level of solubility, masking the taste of Compound 1 in an oral solution is challenging. Even high level of sucralose was evaluated and taste of the oral solution remained bitter. It was found that the taste of sodium benzoate in solution can also be interpreted by some volunteers as bitter. Hence, sodium benzoate was replaced with potassium sorbate. Furthermore, strawberry flavor was chosen, because patients like its sweet taste and it has a good solubility in water. Orange juice or lime juice may be used to dilute the oral solution and to mask the bitter taste before administration.

II. Optimization of Concentration of Citric Acid

Citric acid was used to improve the stability of the formulation and its optimization was studied. The pH value of the solution was a critical parameter which will directly affect the quality of the oral solution, the relationship between the concentration of citric acid in the formula and the initial pH value of the solution was studied systematically.

Based on excipient compatibility study results, the first prototype trial batch was designed with lower concentration of citric acid (from 1.0% w/v to 0.5% w/v) to improve the pH value of the solution. The formula composition and analytical results are described below in Table 4.

TABLE 4
Formula composition of prototype trial batch.

| | Composition with 0.5% w/v citric acid | |
|---|---|---|
| Ingredient Name | Quantity mg/ml | % w/v |
| Drug (API) | 10.0 | 1.0 |
| Methyl paraben | 2.0 | 0.2 |
| Potassium sorbate | 2.0 | 0.2 |
| Sucralose | 40.0 | 4.0 |
| Strawberry flavor (dry) | 10.0 | 1.0 |
| Propylene Glycol | 150.0 | 15.0 |
| Citric acid (anhydrous | 5.0 | 0.5 |
| powder) | | |
| Milli-Q water | Quantity Sufficient to | ~78.1 |
| | produce 1 mL | |
| Total | | 100.0 |
| Final volume of solution/mL | | 100.0 |

For the composition with 0.5% w/v citric acid, the % drug content in the formulation has not a notable decrease after keeping for 1 week at both room temperature and 40° C. The stability of the solution remained satisfactory. However, although the concentration of citric acid was decreased from 1.0% w/v to 0.5% w/v, the pH value was not increased enough as expected (above 5.5). Furthermore, the pH value has a decrease tendency for both room temperature and 40° C. after 1 week.

Further trial batches were designed with 0.2%, 0.1% w/v citric acid and without citric acid, respectively. For the compositions with 0.2%, 0.1% and 0% w/v citric acid, all the pH values of the above three batches at initial time point were above 5.5. Based on previous batch, there was a decrease tendency of pH value for the solution when keeping for a long time. Therefore, a new batch with 0.15% w/v citric acid was prepared. The assay values of the above batches were satisfactory. Results are shown in Tables 5 and 6.

TABLE 5
Formula Composition with 0.15% w/v citric acid.

| | Composition with 0.15% w/v citric acid | | | | |
|---|---|---|---|---|---|
| | Quantity | | Batch | Excess | Disp. |
| Ingredient Name | mg/ml | % w/v | Formula (g) | quantity (%) | Qty (g) |
| Drug (API) | 10 | 1.0 | 1.0 | 1.0 | |
| Methyl paraben | 2 | 0.2 | 0.2 | 100 | 0.4 |
| Potassium sorbate | 2 | 0.2 | 0.2 | 100 | 0.4 |
| Sucralose | 40 | 4.0 | 4.0 | 100 | 8.0 |
| Strawberry flavor (starting as | 10 | 1.0 | 1.0 | 100 | 2.0 |
| dry powder) | | | | | |
| Propylene Glycol | 150 | 15.0 | 15.0 | | 15.0 |
| Citric acid (starting as | 1.5 | 0.15 | 0.15 | 100 | 0.3 |
| anhydrous powder) | | | | | |
| Milli-Q water | Quantity | ~78.45 | 78.45 | | 78.45 |
| | Sufficient to | | | | |
| | produce 1 mL | | | | |
| Total | | 100.0 | 100.0 | | 105.55 |
| Final volume of solution/mL | | | | | 100.0 |

TABLE 6
The analytical results of compositions with 0.15% w/v citric acid.

| Condition | Assay (%) | pH value |
|---|---|---|
| Initial | 102.0 | 5.94 |

Manufacturing Process Development

Based on the experience from excipients compatibility study, methyl paraben has a solubility problem at room temperature. Thus, it was dissolved with 50° C. Milli-Q water first and then cooled down to room temperature. Propylene glycol, other excipients and API were dissolved in the solution successively, and all the clear solution was transferred to volumetric flask to make a target volume. The stirring speed and stirring time during all the steps were finalized after several trail batches.

The manufacturing process includes: methyl paraben, potassium sorbate, strawberry flavor, sucralose and citric acid anhydrous powder were sieved one by one through 40 mesh screen. Methyl paraben was dissolved in a 1000 mL beaker with about 350 mL 50° C. purified water. The solution was stirred at 350 RPM for 10 min at 50° C. Then the solution was stirred without heating until it cooled down to room temperature. Propylene glycol was added and then other excipients were added into the beaker. The mixture was stirred at room temperature at 150 RPM for 10 min. API was added into the beaker. The mixture was stirred at room temperature at 150 RPM for 5 min. After API was dissolved completely, the solution was stirred for 5 min more to make sure a clear solution was obtained. The clear solution was transferred into a 500 mL volumetric flask with glass rod. The beaker and the stirring tool were rinsed with purified water several times. The rinsed solution was also transferred to the 500 mL volumetric flask. Additional purified water was added to the volumetric flask to reach 500 mL volume.

Tech Stability Batch

Based on formulation and manufacturing process development above, tech stability batch was manufactured. The stability samples were putted in 5° C., 25° C./60% relative humidity (RH) for 1 week, 2 weeks and 4 weeks for in-use stability study, while 5° C., 25° C./60% RH and 40° C./75% RH for 1 month, 3 months and 6 months for stability study. Appearance, assay, organic impurity, pH and specific gravity were tested for all the samples at all time-points except 6-month samples for 40° C./75% RH condition.

Summary

After several trial batches, the formula was finalized with suitable pH vale. Based on physical and analytical results from tech stability batch, the formula and manufacturing process of 10 mg/mL oral solution of Compound 1 can be used for future clinical trial manufacturing. The pharmaceutical composition comprises components in Table 5: Compound 1, methyl paraben, potassium sorbate, sucralose, strawberry flavor, propylene glycol, citric acid and water. The pharmaceutical composition has a pH of above 5.5, e.g., around 6.0. The oral pharmaceutical composition comprises Compound 1 at about 10 mg/mL, methyl paraben at about 2 mg/mL, potassium sorbate at about 2 mg/mL, sucralose at about 40 mg/mL, strawberry dry flavor at about 10 mg/mL, propylene glycol at about 150 mg/mL, citric acid at about 1.5 mg/mL, and water.

### Example 6: Development of Flavor System

Evaluation of the above oral solution formulation was conducted by clinical taste testing. To improve flavor quality (palatability) the sweetener system of the flavored Compound 1 solution was modified to reduce its bitterness profile and the aromatic system changed to improve overall flavor quality. Both propylene glycol and methyl paraben contribute aversive sensory attributes; therefore, they were removed and alternatives evaluated. The excipient base system was revised to accommodate the change in the composition components.

To improve flavor quality, propylene glycol was eliminated and the pH lowered to avoid use of parabens preservative. The first step was to reevaluate the amount of citric acid present in the excipient base. As citric acid was increased (from 0.3% to 0.5%), the balance of sour and bitter basic tastes improved. This effect was optimal at 0.5%, and expected to be appropriate to support the addition of other flavoring excipients. Accordingly, a buffer system containing 0.5% citric acid was advanced for further white base development.

Next, the addition of sweeteners was reevaluated. Four candidates: sodium saccharin, acesulfame potassium, neotame, and sucralose were screened. While many of the candidate sweeteners failed to produce optimal results, sucralose was evaluated up to 0.55%. A concentration of 0.5% was determined to be most effective at reducing mimetic bitterness, becoming more bitter at higher strengths. Accordingly, sucralose at 0.5% was selected for advancement.

Additional flavors were evaluated. The raspberry and grape flavor systems both provided good coverage of the bitterness, with no bitter breakthrough. The two flavored Compound 1 formulations are reasonably high in overall flavor quality.

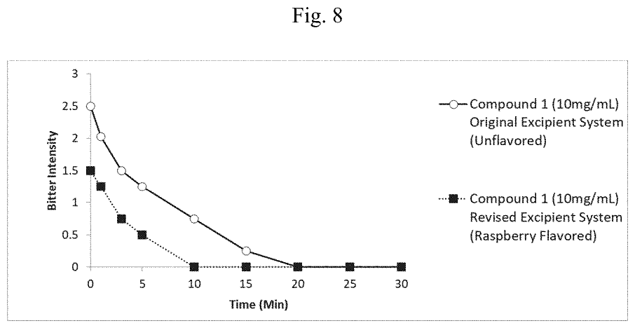

The resulting formulation was then retested by clinical taste testing and the resulting improvements in flavor profile identified in FIG. 8. The bitterness of the Compound 1 is reduced in the improved revised excipient base formulation with the addition of raspberry flavor over the unflavored original excipient base composition. The resulting bitterness is somewhat above the target (1-intensity) but is well blended by the complementary sweet and sour basic tastes.

The improved pharmaceutical composition components are listed in Table 7.

TABLE 7
Pharmaceutical composition components post Flavor System.

| | | Quantity (g/100 mL) | Quantity (g/100 mL) |
|---|---|---|---|
| No. | Material | Grape | Raspberry |
| 1 | Compound 1 (API) | 1.0 g | 1.0 g |
| 2 | Potassium Sorbate | 0.2 g | 0.2 g |
| 3 | Sucralose | 0.5 g | 0.5 g |
| 4 | Citric acid | 0.5 g | 0.5 g |
| 5 | Grape flavor | 0.38 g | N/A |
| 6 | Raspberry flavor | N/A | 0.3 g |
| 7 | Purified water | QS to 100 mL | QS to 100 mL |

### Manufacturing Process Development

The manufacturing process includes: Compound 1 (API), potassium sorbate, flavor, sucralose, and citric acid. The API and each excipient were weighed out and added to a stirred vessel containing 80% of the targeted volume of water. The mix was left to stir for 10 minutes after which time a clear solution had formed. The resulting solution was diluted to final volume and transferred to a sealed storage container

A batch formula was finalized with a suitable taste mask of the API, minimal adverse flavors from the excipient package, and a suitable pH value that allowed for preservative effectiveness and desirable flavor profile when used with the selected flavoring agents. Accelerated and real time stability testing indicated the formulation could be used for clinical trials.

The oral pharmaceutical composition includes Compound 1, potassium sorbate, sucralose, flavor, citric acid and water. The pharmaceutical composition has a pH of 3.0 to 6.0. The oral pharmaceutical composition comprises Compound 1 at about 10.0 mg/mL, potassium sorbate at about 2.0 mg/mL, sucralose at about 5.0 mg/mL, either grape flavor at about 3.8 mg/mL or raspberry flavor at about 3.0 mg/mL, citric acid at about 5.0 mg/mL, and water.

What is claimed is:

1. An pharmaceutical composition comprising:
(i) about 10.0 mg/mL of 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or hydrate thereof; and
(ii) an excipient base comprising about 2.0 mg/mL of potassium sorbate, about 5.0 mg/mL sucralose, about 5.0 mg/mL citric acid, or any combination thereof, wherein the pharmaceutical composition is in the form of an oral solution suitable for administration to a patient.

2. The pharmaceutical composition of claim 1, further comprising a flavor.

3. The pharmaceutical composition of claim 1 or claim 2, wherein the flavor is a cherry flavor, a raspberry flavor, a grape flavor, a strawberry flavor, or a tutti-fruity flavor.

4. The pharmaceutical composition of any one of claims 1 to 3, wherein the flavor is a grape flavor.

5. The pharmaceutical composition of any one of claims 1 to 3, wherein the flavor is a raspberry flavor.

6. The pharmaceutical composition of claim 5, wherein the composition comprises about 3.0 mg/mL of a raspberry flavor.

7. The pharmaceutical composition of any one of claims 1 to 6, wherein the pH of the pharmaceutical composition is from about 3.0 to about 6.0, or from about 5.5 to about 6.5.

8. The pharmaceutical composition of any one of claims 1 to 6, wherein the pH of the pharmaceutical composition is above 5.5.

9. The pharmaceutical composition of claim 7, wherein the pH of the pharmaceutical composition is about 5.5, about 5.6, about 5.7, about 5.8, about 5.9, about 6.0, about 6.1, about 6.2, about 6.3, about 6.4, or about 6.5.

10. A pharmaceutical composition comprising: 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or hydrate thereof; and an excipient base, wherein the composition is in the form of an oral liquid solution.

11. The pharmaceutical composition of claim 10, comprising from about 5.0 mg/mL to about 15.0 mg/mL of Compound 1, or a pharmaceutically acceptable salt, solvate, or hydrate thereof.

12. The pharmaceutical composition of claim 10, comprising about 6.0 mg/mL, about 7.0 mg/mL, about 8.0 mg/mL, about 9.0 mg/mL, about 10.0 mg/mL, about 11.0 mg/mL, about 12.0 mg/mL, or about 13.0 mg/mL of Compound 1, or a pharmaceutically acceptable salt, solvate, or hydrate thereof.

13. The pharmaceutical composition of any one of claims 10 to 12, wherein the excipient base comprises potassium sorbate, sucralose, citric acid, or any combination thereof.

14. The pharmaceutical composition of claim 13, wherein the excipient base comprises from about 1.0 mg/mL to about 3.0 mg/mL of potassium sorbate.

15. The pharmaceutical composition of claim 13, wherein the excipient base comprises about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, or about 3.0 mg/mL of potassium sorbate.

16. The pharmaceutical composition of claim 13, wherein the excipient base comprises about 0.2% w/v of potassium sorbate.

17. The pharmaceutical composition of claim 13, wherein the excipient base comprises from about 1.0 mg/mL to about 20.0 mg/mL of sucralose.

18. The pharmaceutical composition of claim 13, wherein the excipient base comprises about 1.0 mg/mL, about 5.0 mg/mL, about 10.0 mg/mL, about 15.0 mg/mL or about 20.0 mg/mL of sucralose.

19. The pharmaceutical composition of claim 13, wherein the excipient base comprises about 0.50% w/v of sucralose.

20. The pharmaceutical composition of claim 13, wherein the excipient base comprises from about 1.0 mg/mL to about 10.0 mg/mL of citric acid.

21. The pharmaceutical composition of claim 13, wherein the excipient base comprises about 2.0 mg/mL, about 2.5 mg/mL, about 3.0 mg/mL, about 3.5 mg/mL, about 4.0 mg/mL, about 4.5 mg/mL, about 5.0 mg/mL, about 5.5 mg/mL, or about 6.0 mg/mL of citric acid.

22. The pharmaceutical composition of claim 13, wherein the excipient base comprises about 0.5% w/v of citric acid.

23. The pharmaceutical composition of any one of claims 10 to 22, wherein the excipient base comprises about 2.0 mg/mL potassium sorbate, about 5.0 mg/mL sucralose, about 5.0 mg/mL of citric acid, or any combination thereof.

24. The pharmaceutical composition of any one of claims 10 to 23, further comprising a flavor.

25. The pharmaceutical composition of claim 24, wherein the flavor is a cherry flavor, a grape flavor, a raspberry a strawberry flavor, or a tutti-fruity flavor.

26. The pharmaceutical composition of claim 24 or 25, wherein the flavor is a grape flavor.

27. The pharmaceutical composition of claim 24 or 25, wherein the flavor is a raspberry flavor.

28. The pharmaceutical composition of any one of claims 24 to 27, further comprising from about 1.0 mg/mL to about 5.0 mg/mL of a flavor.

29. The pharmaceutical composition of claim 28, wherein the composition further comprises about 3.0 mg/mL of a raspberry flavor.

30. The pharmaceutical composition of claim 29, wherein the raspberry flavor is Sensient Natural and Artificial Raspberry Flavor, SN1000073269.

31. The pharmaceutical composition of any one of claims 10 to 30, wherein the pH of the pharmaceutical composition is from about 3.0 to about 6.0, or from about 5.5 to about 6.5.

32. The pharmaceutical composition of claim 31, wherein the pH of the pharmaceutical composition is about 5.5, about 5.6, about 5.7, about 5.8, about 5.9, about 6.0, about 6.1, about 6.2, about 6.3, about 6.4, or about 6.5.

33. The pharmaceutical composition of any one of claims 10 to 30, wherein the pH of the pharmaceutical composition is above 5.5.

34. The pharmaceutical composition of any one of claims 1 to 33, wherein the composition is formulated for pediatric use.

35. A method for treating sickle cell disease in a subject in need, comprising administering a therapeutically effective amount of the pharmaceutical composition of any one of claims 1 to 34.

36. The method of claim 35, wherein the pharmaceutical composition is taken with food.

37. The method of claim 35, wherein the pharmaceutical composition is administered once per day, twice per day, or three times per day.

38. The method of claim 35, wherein the pharmaceutical composition is administered once per day.

39. The method of any one of claims 35 to 38, wherein the pharmaceutical composition is administered for at least 4 weeks, 12 weeks, 16 weeks, or 24 weeks.

40. The method of any one of claims 35 to 39, further comprising administering hydroxyurea (HU).

41. The method of any one of claims 35 to 40, comprising administering to the subject about 0.3 mg/kg to about 6.0 mg/kg or from about 0.3 mg/kg to about 1.0 mg/kg of subject's body mass per day or per dose of Compound 1, or a pharmaceutically acceptable salt, solvate, or hydrate thereof.

42. The method of any one of claims 35 to 41, wherein the patient in need thereof is a pediatric patient.

43. A method for treating sickle-β⁰ thalassemia in a subject in need, comprising administering a therapeutically effective amount of the pharmaceutical composition of any one of claims 1 to 34.
